(19) United States
(12) Patent Application Publication
(10) Pub. No.: US 2026/0165833 A1
(43) Pub. Date: Jun. 18, 2026

(54) ADJUSTABLE HEART VALVE

(71) Applicant: MDF Holding Co., LLC, Spokane, WA (US)

(72) Inventor: Matthew D. Forrester, Spokane, WA (US)

(21) Appl. No.: 19/388,136

(22) Filed: Nov. 13, 2025

Related U.S. Application Data
(60) Provisional application No. 63/788,262, filed on Apr. 14, 2025.
(60) Provisional application No. 63/722,131, filed on Nov. 19, 2024.

(51) Int. Cl. A61F 2/24

(52) U.S. Cl. CPC A61F 2/2412; A61F 2230/0065; A61F 2250/001; A61F 2250/0018

(57) ABSTRACT

A valve includes a base having one or more bands, a frame coupled to the base, leaflets coupled to the frame, and an adjustment mechanism to adjust a size of the valve.

### CROSS-REFERENCE TO RELATED APPLICATIONS

This application claims priority to U.S. Provisional Application No. 63/722,131, filed Nov. 19, 2024, entitled “Adjustable Valve,” and U.S. Provisional Application No. 63/788,262, filed Apr. 14, 2025, entitled “Adjustable Valve,” the entirety of which are herein incorporated by reference.

### BACKGROUND

Accurate prosthesis sizing is critical in surgical aortic valve replacement (SAVR), particularly with regard to matching the prosthetic valve to the dimensions of the native aortic annulus. Undersizing may lead to patient prosthesis mismatch (PPM), resulting in elevated transvalvular gradients, impaired hemodynamics, and reduced long-term valve durability. Moreover, an undersized surgical valve may significantly limit the feasibility and efficacy of subsequent valve-in-valve transcatheter aortic valve replacement (ViV-TAVR), due to restricted internal diameter and constrained expansion of the transcatheter valve.

Annular enlargement techniques have been developed to facilitate the implantation of larger prosthetic valves, with the goal of optimizing hemodynamic performance and preserving future transcatheter treatment options. Traditional surgical approaches, such as the Nicks, Manouguian, or Konno-Rastan procedures, employ patch augmentation of the aortic annulus or aortic root to achieve increased annular diameter. While effective, these procedures are technically demanding, prolong cardiopulmonary bypass and cross-clamp times, and are associated with increased perioperative morbidity, including bleeding, conduction system injury, and complex anatomical reconstruction. Additionally, patch augmentation may result in non-anatomical geometries that adversely affect prosthetic valve function and durability.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features. The systems depicted in the accompanying figures are not to scale and components within the figures may be depicted not to scale with each other.

FIG. 1 illustrates an example valve that is adjustable in size, according to an example of the present disclosure.

FIGS. 2A and 2B illustrate isometric views of the valve of FIG. 1, showing an outer layer (e.g., fabric) of the valve removed to illustrate example components of the valve, according to an example of the present disclosure.

FIG. 3 illustrates an example expansion of the valve of FIG. 1, according to an example of the present disclosure.

FIGS. 4A-4D illustrate various side views of the valve of FIG. 1, showing an outer layer (e.g., fabric) removed to illustrate example components of the valve, according to an example of the present disclosure.

FIGS. 5A and 5B illustrate various views of example components of the valve of FIG. 1, such as an example base, example bands, and an example adjustment mechanism, according to an example of the present disclosure.

FIGS. 6A-6C illustrate an example first band of the valve of FIG. 1, according to an example of the present disclosure.

FIGS. 7A-7C illustrate an example second band of the valve of FIG. 1, according to an example of the present disclosure.

FIGS. 8A and 8B illustrate an example third band of the valve of FIG. 1, according to an example of the present disclosure.

FIGS. 9A and 9B illustrate an example overlap, or expansion of an example base of the valve of FIG. 1, according to an example of the present disclosure.

FIGS. 10-13 illustrate an example sequence to expand the valve of FIG. 1, according to an example of the present disclosure.

FIG. 14 illustrates isometric views of the valve of FIG. 1 during expansion, according to an example of the present disclosure.

FIG. 15 illustrates an isometric view of an example valve that is adjustable in size, according to an example of the present disclosure.

FIGS. 16A and 16B illustrate an example frame of the valve of FIG. 15, according to an example of the present disclosure.

FIGS. 17A and 17B illustrate side views of the valve of FIG. 15, according to an example of the present disclosure.

FIGS. 18A and 18B illustrate an example sequence to expand the valve of FIG. 15, according to an example of the present disclosure.

FIG. 19 illustrates an isometric view of an example valve that is adjustable in size, according to an example of the present disclosure.

FIGS. 20A and 20B illustrate an example adjustment mechanism of the valve of FIG. 19, according to an example of the present disclosure.

FIGS. 21A and 21B illustrate an example adjustment of the valve of FIG. 19 via the adjustment mechanism of FIGS. 20A-20B, according to an example of the present disclosure.

FIG. 22 illustrates an example coupling between a base and a frame of the valve of FIG. 19, according to an example of the present disclosure.

FIG. 23 illustrates an example adjustment to the base of the valve of FIG. 19, according to an example of the present disclosure.

FIG. 24 illustrates an example brace that is usable with the valve of FIG. 1, the valve of FIG. 15, and/or the valve of FIG. 19, according to an example of the present disclosure.

FIG. 25 illustrates the brace of FIG. 24 engaged, or disposed around, the valve of FIG. 1, according to an example of the present disclosure.

### DETAILED DESCRIPTION

This application is directed, at least in part in part, to a prosthetic adjustable heart valve (herein, “valve”) designed for use in transcatheter aortic valve replacement (TAVR), surgical aortic valve replacement (SAVR), or other cardiac valve replacement procedures. In some instances, the valve is dynamically adjustable in size, such as in diameter or cross-sectional area, to accommodate future valve-in-valve (ViV) procedures and/or at the time of implantation. The valve may include a frame supporting a set of prosthetic leaflets and one or more integrated adjustment mechanism(s) operable to modify the valve’s size. Actuation of the adjustment mechanism(s) may induce radial expansion of the frame, effectively increasing one or more of the valve’s dimensions. The leaflets are engineered with redundant coaptation zones, allowing them to maintain functional integrity and prevent regurgitation across varying diameters. This adjustable architecture enhances clinical flexibility, facilitates future interventions, and may mitigate complications related to undersized prosthetic valves.

The valve, as described herein, may be designed for implantation within the aortic position, such as in transcatheter or surgical aortic valve replacement procedures. However, the structural and functional features of the valve may render it suitable for use in other cardiac valve positions or related applications. For example, the valve may be adapted for deployment in the mitral, tricuspid, or pulmonary positions, either through surgical implantation or transcatheter approaches, with appropriate modifications to the frame geometry, leaflet design, or anchoring mechanisms to accommodate the anatomical and hemodynamic differences of each location. Furthermore, the valve’s adjustable design and expansion capabilities may find use in non-valvular cardiovascular applications, such as annular remodeling devices, conduit replacements, or staged structural heart interventions.

In some instances, and as will be explained herein, the valve includes a structural coupling between a base, the frame, and one or more struts, which together form an integrated assembly capable of transmitting mechanical forces during valve expansion or actuation. The base serves as the foundational support structure, which may include one or more circumferential bands, and provides the anchoring platform for the valve within a native annulus of a patient. Extending from the base are the struts, which function as mechanical linkages or load-bearing members that connect the base to the frame. The frame, which supports the leaflets and defines the valve’s effective orifice, is mechanically coupled to the struts. This coupling permits radial forces generated at the base, for instance via the adjustment mechanism(s) or one or more biasing element(s), to be efficiently transmitted through the struts to the frame, resulting in synchronous radial expansion of the valve structure.

The valve may include the posts (e.g., columns, pillars, etc.) configured to support the leaflets. In some instances, the frame includes three posts, such as a first post, a second post, and a third post. The posts may be connected together and formed from a piece of bent material. In some instances, the first post supports the first leaflet and the second leaflet, the second post supports the second leaflet and the third leaflet, and the third post supports the first leaflet and the third leaflet. The leaflets may be coupled to the frame through surgical-grade sutures, adhesives, or other fixation techniques (e.g., ultrasonic welding).

The frame is mechanically coupled to the base via the struts (e.g., rods, support beams, columns, etc.), which serve to transmit forces between the base and the frame. The struts may be directly or indirectly connected to either the base or the frame, optionally through intermediate structural members. The base itself may be formed from the circumferential bands (e.g., continuous rings or arcuate segments), which may interlock, overlap, or otherwise engage with one another. For example, the base may be formed from three bands, such as a first band, a second band, and a third base. However, although described as including three bands, alternative configurations with more or fewer bands are also contemplated.

The base may define an orifice through which blood flows. During expansion of the valve, an internal diameter of the valve is increased. The orifice of the valve may be adjusted as a result of expansion. A fabric of the valve, which may enclose or surround components of the valve, may permit such expansion, or stated alternatively, may include sufficient give, slack, etc., so as to not restrict a desired expansion.

In some instances, the adjustment mechanism(s) is integrated into the base and may be formed wholly or partially from the bands. For example, the adjustment mechanism(s) may incorporate a buckle (e.g., tab, hook, or clasp) configured to engage with one or more receptacles (e.g., notches, channels, or slots) on one or more bands. By selecting a specific receptacle for engagement, the buckle alters the size of the base and, via the struts, adjusts the overall diameter of the valve. More particularly, a radial force exerted by the bands during actuation is transferred to the frame, resulting in expansion. The mechanical interlock between the buckle and receptacle not only secures the bands in a fixed position but also stabilizes the expanded configuration of the valve. For example, an engagement between the buckle and the receptacle may prevent unintended expansion or constriction of the valve.

Positioning the buckle within available receptacles may determine the degree by which the valve is expanded. For example, placement in a first receptacle may yield a valve diameter of approximately 21 mm, while engagement with a second receptacle may increase the diameter to 25 mm. Multiple receptacles may be included to allow for graduated or incremental expansion. Additionally, more than one buckle-receptacle pair or multiple adjustable bands may be implemented to facilitate multi-point size modulation and ensure circumferential symmetry during expansion. For example, a buckle disposed on the first band may engage within a receptacle of the second band, a buckle disposed on the second band may engage with a receptacle of the third band, and a buckle disposed on the third band may engage with a receptacle of the first band. Doing so may ensure that the valve is uniformly expanded and/or exerts uniform pressure on the native annulus.

The valve may be expanded in situ via various actuation methods. As one non-limiting example, a transcatheter balloon may be introduced post-implantation and used to exert outward radial force on the base. As the balloon inflates, the buckle disengages from one receptacle and engages a subsequent one, resulting in a controlled expansion of the frame. In other instances, as the ballon inflates, the buckle engages with a receptacle. For example, at a time of implantation, the buckle may not be disposed with, or engaged with, the receptacle, respectively. After or during implantation, to increase the size of the valve, the balloon may be inflated to engage the buckles with the receptacles, respectively. The mechanical linkage between the base and the frame via the struts ensures synchronized expansion. Moreover, the buckle may be engineered to require a threshold activation force, preventing premature disengagement and ensuring stable deployment. Upon reaching the predetermined diameter, or when the buckle engages with a desired receptacle, the balloon is deflated and removed. The disposition of the buckle within the receptacle maintains the valve in the expanded state.

Alternative adjustment mechanisms may be employed in place of, or in addition to, the buckle and receptacle style mechanism described above. These may include, without limitation, ratcheting systems, locking clasps, expanding latches, or other mechanical interlocks. The mechanism may also feature male-female connectors, prongs and slots, gear-like teeth, or telescoping features, which provide secure, reversible or one-way size modulation. Actuation may occur via a variety of stimuli or tools, including magnetic fields, mechanical drivers, balloon catheters, thermal stimuli, or light-based (e.g., UV) activation. Adjustment mechanisms may be located on the base, the frame, the bands, or any combination thereof and may be designed for intravascular or extravascular access, depending on the procedural approach.

In some instances, a temporary constraining device or brace (e.g., clamp, cage, retainer, etc.) may be deployed around an outer surface of the valve during delivery to inhibit premature expansion. Once the valve is correctly positioned within the annulus, the brace may be removed, allowing controlled expansion of the frame to its intended diameter. For example, once the valve is disposed within the native annulus, the brace may be removed and therein, the valve may be expanded using the methods described above.

In some instances, the base or the adjustment mechanism may incorporate one or more biasing elements (e.g., springs, elastic bands, memory materials, etc.). In some instances, the biasing elements may be interposed between or extend between one or more of the bands. For example, a first biasing element may be positioned between an end of the first band and an end of the second band, while a second biasing element may be positioned between an end of the second band and an end of the third band. The biasing elements may be engineered to store elastic or mechanical energy, gradually releasing it over time or in response to specific stimuli, such as temperature, mechanical pressure, or photonic activation, thereby inducing progressive annular dilation. The biasing elements may act independently or synergistically with the adjustment mechanism to facilitate expansion.

Post-implantation, the biasing elements may apply a continuous or staged outward radial force to the base, which is transmitted to the frame via the struts. The biasing elements allow the frame to expand and exert gentle, sustained pressure on the surrounding annular tissue, potentially promoting tissue remodeling and annular compliance. In some instances, symmetric placement of multiple biasing elements ensures uniform expansion and preserves valve geometry. The biasing elements may be used either in conjunction with or as alternatives to the adjustment mechanism as discussed above. Moreover, the brace, as described above, may be used to constrain expansion of the biasing elements. However, once the brace is removed, the biasing elements are configured to exert forces to expand the size of the valve.

In some instances, the biasing elements may include coil springs, helical or flat springs, or elastic polymeric bands. The force profile of the biasing elements may be tailored by modifying parameters such as coil density, cross-sectional geometry, material modulus, and unloaded length. In some instances, the biasing elements may include stimuli-responsive materials (e.g., shape-memory polymers, thermoplastics, alloys, etc.), which expand in response to specific inputs such as UV light or body temperature. These materials may be integrated into the bands, base, or structural elements of the frame.

The leaflets are designed with redundant coaptation zones to maintain valve competence across multiple diameters of the valve. During radial expansion, the posts separate (e.g., move radially outward from one another), thereby stretching the leaflets. The leaflet geometry is pre-configured to preserve effective leaflet coaptation even in expanded states. However, initial implantation may feature deliberate leaflet redundancy, which is then utilized during future expansions to maintain coaptation and valve competence.

In some instances, the adjustment mechanism may be omitted, and expansion may be achieved solely through biasing elements that gradually exert radial force over time. For example, the buckle-style adjustment mechanism as described above may be excluded, and valve expansion may be governed by passive or active biasing systems, which expand the valve without discrete mechanical locking steps. Alternatively, the biasing elements may be omitted and the valve may be adjusted in size solely through one or more of the buckle-style adjustment mechanism. Although the valve is described with certain components or having a certain function, other embodiments are envisioned.

The valve may be fabricated from biocompatible, deformable materials such as cobalt-chromium alloys, nitinol, stainless steel, or composite materials, which allow for elastic deformation during expansion. The posts, the base, the biasing elements, etc., may be encased or covered with the fabric (e.g., woven or knitted polyester, expanded polytetrafluoroethylene, etc.) that permits suturing of the valve to the patient. For example, the fabric may be sewn to the leaflets and/or wrapped around the posts. Sutures may be used to attach the valve to native tissue, and the fabric may also serve to encapsulate or protect the adjustment mechanism, thereby maintaining structural coherence. In some instances, the frame may be partially or fully encased in a fabric covering, which may assist in securing the leaflets, promoting tissue ingrowth, or enhancing sealing. The leaflets may be constructed from biological tissue (e.g., bovine pericardium) or synthetic polymers. The valve, in general, is manufactured from biocompatible, durable, and hemocompatible materials that are capable of resisting calcification and fatigue under constant cyclic loading.

Accordingly, the disclosure describes an adjustable valve designed for both surgical and transcatheter applications, enabling broad clinical utility. The valve may include adjustment mechanisms for instantaneous expansion at the time of implantation, utilizing multiple engagement points to achieve optimal sizing, and may be engineered to allow for a second-stage augmentation at a later date, thereby supporting staged enlargement strategies. In addition, the valve may include biasing elements—such as springs or shape-memory alloys—that may gradually expand the valve over a period of weeks, promoting tissue remodeling and annular compliance. The leaflet architecture is optimized to begin with redundant coaptation, transitioning to optimal coaptation as the valve expands, a process facilitated by a frame that reduces in height and broadens at the interleaflet triangles, while also allowing for alternative engineering solutions. To ensure procedural control, an external constraining device may prevent premature expansion during delivery, which is removed to permit controlled actuation of the adjustment mechanisms.

The ability to expand the valve either instantaneously at implantation or in a staged manner at later time points offers significant clinical advantages across a range of patient populations. For example, staged or gradual expansion may be particularly beneficial for patients with small aortic annuli, where initial conservative sizing may minimize procedural risk and allow for subsequent enlargement as anatomical conditions permit. This approach is also advantageous in pediatric or younger adult patients, who may require future ViV procedures as their anatomy changes over time. Gradual expansion using springs or shape-memory alloys can promote tissue remodeling and annular compliance, potentially reducing the risk of paravalvular leak and improving long-term hemodynamic performance. By enabling both immediate and delayed augmentation of valve diameter, the system provides clinicians with enhanced flexibility to tailor therapy to individual patient needs and evolving clinical scenarios.

The present disclosure provides an overall understanding of the principles of the structure, function, device, and system disclosed herein. One or more examples of the present disclosure are illustrated in the accompanying drawings. Those of ordinary skill in the art will understand and appreciate that the devices, the systems, and/or the methods specifically described herein and illustrated in the accompanying drawings are non-limiting embodiments. The features illustrated or described in connection with one embodiment or instance may be combined with the features of other embodiments or instances. Such modifications and variations are intended to be included within the scope of the disclosure and appended claims.

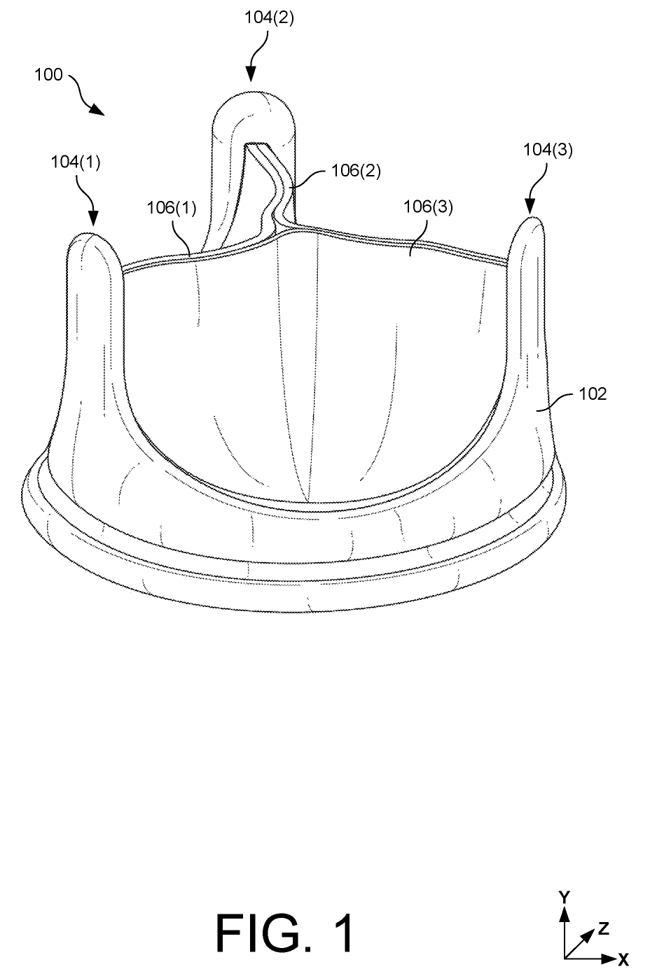

FIG. 1 illustrates an isometric view of an example valve 100, according to an example of the present disclosure. In some instances, the valve 100 includes a frame with posts 104, such as a first post 104(1), a second post 104(2), and a third post 104(3). The posts 104 support one or more leaflets 106, such as a first leaflet 106(1), a second leaflet 106(2), and a third leaflet 106(3). In some instances, each of the posts 104 serves as a support between two adjacent leaflets 106. For example, the first post 104(1) may support the first leaflet 106(1) and the second leaflet 106(2); the second post 104(2) may support the second leaflet 106(2) and the third leaflet 106(3); and the third post 104(3) may support the third leaflet 106(3) and the first leaflet 106(1). As will be explained herein, this configuration facilitates symmetric leaflet motion and proper coaptation throughout the cardiac cycle, allowing the valve 100 to replicate native valve biomechanics.

As will be explained herein, the valve 100 is configured to expand radially (e.g., in the X-Z plane) to accommodate varying annular sizes. In some instances, the valve 100 is expanded at or during the time of implantation, or at future instances in time (e.g., several weeks, months, years, etc.) to allow for future ViV procedures. Expansion of the valve 100 may be achieved through mechanical actuation, balloon dilation, or activation of internal biasing elements, resulting in outward displacement of the posts 104. As the posts 104 move outward (e.g., in the X and/or Z direction), the frame enlarges in diameter (e.g., about the X-Z plane) while maintaining proper alignment and support for the leaflets 106. Moreover, when expanded, the posts 104 maintain their relative orientation to preserve leaflet geometry and ensure reliable hemodynamic performance.

Portions of the valve 100, such as the frame, the posts 104, and/or portions of the leaflets 106 are at least partially covered in a fabric 102. The fabric 102 may serve several purposes, including securing the leaflets 106 to the posts 104, promoting endothelialization or tissue ingrowth, and enhancing sealing at the valve-tissue interface to reduce the risk of paravalvular leakage. The fabric 102 also permits the valve 100 to be sutured within a native annulus of a patient. The fabric 102 may be wrapped, bonded, or sutured around the posts 104, and may also extend across other regions of the frame to encapsulate structural elements and minimize direct blood contact with metallic or composite materials. Additionally, the fabric 102 may be designed to accommodate expansion of the valve 100, such as by incorporating pleated, elastic, or compliant regions that unfold or stretch during radial enlargement. In this way, the fabric 102 maintains continuous coverage and leaflet support as the valve 100 transitions to different sizes, either intraoperatively or in post-implantation ViV scenarios.

In some instances, the valve 100 may be utilized in both transcatheter aortic valve replacement (TAVR) and surgical aortic valve replacement (SAVR) procedures. Although primarily described in the context of the aortic position, the valve 100 may also be applicable to other cardiac valve positions, such as the mitral, tricuspid, or pulmonary valves, with appropriate anatomical and functional adaptations. Additionally, while referred to herein as an adjustable valve, the valve 100 may alternatively be described as an expandable valve, a valve configured for dimensional adjustment, or, more broadly, as a cardiovascular implantable device or apparatus capable of structural expansion to accommodate varying anatomical conditions or procedural requirements.

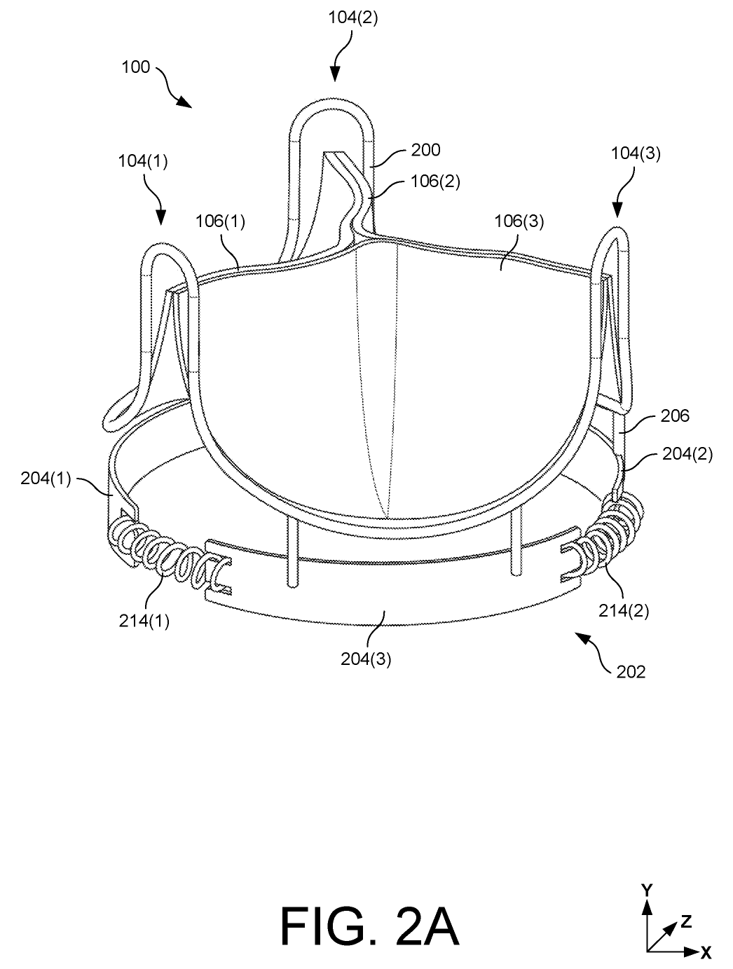

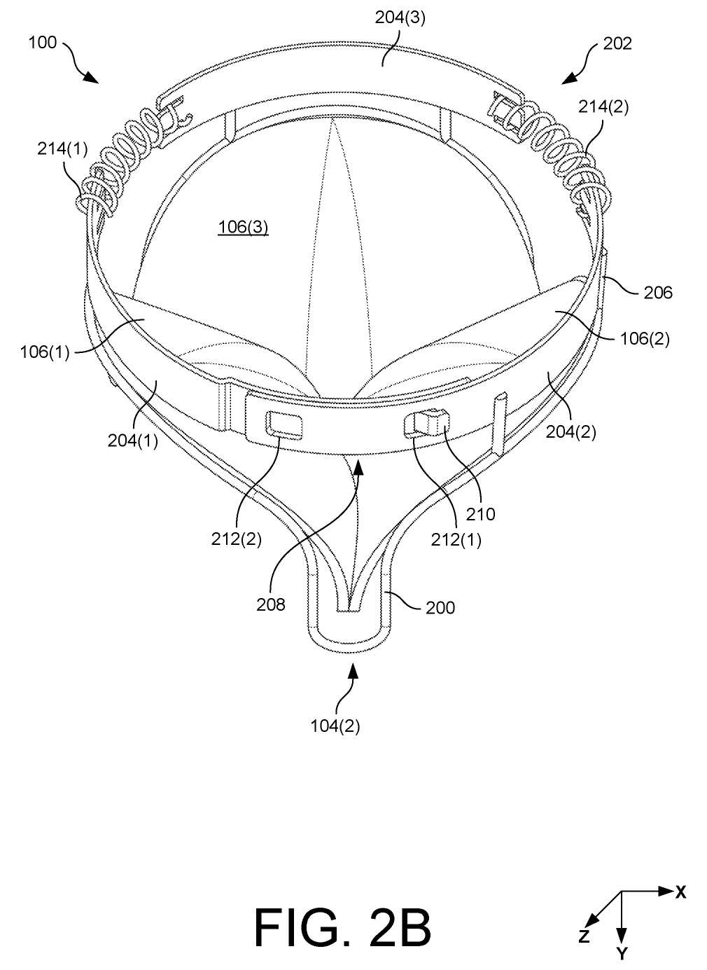

FIGS. 2A and 2B illustrate isometric views of the valve 100, showing the fabric 102 removed, according to an example of the present disclosure. The valve 100 includes a frame 200 that forms the posts 104, such as the first post 104(1), the second post 104(2), and the third post 104(3). As introduced above, the leaflets 106 extend between the posts 104, for example, such that the first leaflet 106(1) extends between the first post 104(1) and the second post 104(2), the second leaflet 106(2) extends between the second post 104(2) and the third post 104(3), and third leaflet 106(3) extends between the first post 104(1) and the third 104(3). The leaflets 106 are securely attached to the posts 104 to ensure proper valve function.

In some instances, the leaflets 106 may be secured to the posts 104 using a variety of methods. For example, the leaflets 106 may be sutured directly to the posts 104, the leaflets 106 may be adhered to the posts 104 using medical-grade adhesives or bonding agents, the leaflets 106 may be mechanically coupled to the posts 104, etc. Moreover, the leaflets 106 may be integrated with the fabric 102, and/or coupled to the posts 104 with the assistance of the fabric 102. Additionally, certain designs may incorporate molding or over-molding processes, whereby a material of the leaflet 106 is formed directly onto or around the posts 104 to create an integral, monolithic structure. Regardless of the specific implementation, the attachment methods maintain leaflet coaptation, accommodate radial expansion of the frame 200, and ensure long-term durability under physiological loading conditions.

The valve 100 includes a base 202 that serves as a foundational ring or structural support positioned separately from the frame 200. The base 202 may include one or more bands 204, such as a first band 204(1), a second band 204(2), and a third band 204(3). The bands 204 form a continuous or segmented ring structure that permits adjustment of the valve 100. Although shown as including three of the bands 204, the base 202 may include more than or less than three of the bands 204.

The frame 200 mechanically couples to the base 202 via one or more struts 206. The struts 206 extend between the portions of the frame 200 and the bands 204 of the base 202. The struts 206 may be representative of any suitable pillar, coupling mechanism, tie, band, etc., that couples the frame 200 to the base 202. In some instances, the struts 206 are formed from the base (202), or the bands 204, and/or the frame 200. In some instances, each of the bands 204 may couple to the frame 200 via two of the struts 206. Moreover, as shown, the frame 200 may couple to the bands 204 at a location between the posts 104, respectively. As will be explained herein, this coupling allows the forces to be transmitted to the frame 200, enabling coordinated radial expansion of the valve 100. Although described as separate components, the frame 200 and the base 202 (and/or the struts 206), may be a single unitary structure or assembly of components. Moreover, although described as coupling to the base 202 via the struts 206, the frame 200 and the base 202 may be coupled together in different manners.

The valve 100 includes an adjustment mechanism 208 that may be selectively adjusted to vary the size of the valve 100. In some instances, the adjustment mechanism 208 is incorporated within the base 202 and/or includes the bands 204. The adjustment mechanism 208 may include the buckle 210 that is moveable between the receptacles 212 to adjust the size of the valve 100. In some instances, the buckle is integrated within one of the bands 204, while the receptacles 212 are integrated within another of the bands 204. For example, as shown in FIG. 2B, the first band 204(1) may include the buckle 210 of the adjustment mechanism 208 and the second band 204(2) may form the receptacles 212 of the adjustment mechanism 208, such as a first receptacle 212(1) and a second receptacle 212(2). The buckle 210 may be moved between the receptacles 212, where each of the receptacles 212 may be associated with a predetermined diameter of the valve 100. For example, movement of the buckle 210 between the receptacles 212 adjusts the circumference of the base 202, which, through the struts 206, causes the frame 200 to radially expand, thereby increasing the internal diameter of the valve 100.

In some instances, the buckle 210 is disposed external to the receptacles 212, whereby the valve 100 may have its smallest diameter, or size (e.g., at a time of implantation). After being placed within the annulus of the patient, for example, the valve 100 may be expanded (as will be described herein) to dispose the buckle 210 within one of the receptacles 212, such as the first receptacle 212(1). Here, the buckle 210 may have a second size that is larger than when implanted. Moreover, at future instances in time, the valve 100 may be expanded, whereby the buckle 210 may move from the first receptacle 212(1) to the second receptacle 212(2), whereby the valve 100 may have a third size.

In some instances, the adjustment mechanism 208 may be actuated in a variety of ways, either during the initial implantation procedure or at a later time point, to selectively expand the diameter of the valve 100. In some instances, expansion is achieved by delivering radial force to the base 202 using a balloon catheter, which is introduced transcatheterally and inflated within the valve 100 to apply outward pressure. This pressure may disengage the buckle 210, for example, from one of the receptacles 212 to another of the receptacles 212 (e.g., from the first receptacle 212(1) to the second receptacle 212(2), during which the diameter of the valve 100 increases. Moreover, the pressure may move the buckle 210 from an unengaged position, to an engaged position whereby the buckle 210 is disposed within the first receptacle 212(1), for example. Alternatively, manual actuation may be performed using catheter-delivered tools that engage with the adjustment mechanism 208, mechanical interfaces, such as keyways, drive slots, or gears, integrated into the bands 204 of the base 202. In certain embodiments, remote actuation may be employed, using magnets, light-activated materials (e.g., UV-sensitive polymers), or thermo-responsive alloys (e.g., shape-memory metals like Nitinol) to trigger expansion when exposed to external stimuli. Once adjusted, the adjustment mechanism 208 may lock into place via integrated stops, detents, or frictional engagement to resist recoil or contraction under hemodynamic loading.

As will be explained herein, although a certain example of the adjustment mechanism 208 is shown, other examples are envisioned. For example, a ratcheting mechanism with a pawl or locking tab may be used, a screw-based mechanism may be used, and so forth. Still, the adjustment mechanism may include tool-actuated elements, such as keyed slots or recessed drivers, allowing for manual expansion. These various adjustment designs can be selectively applied based on procedural needs, desired expansion profile, and anatomic constraints. Moreover, the adjustment mechanism 208 may be located differently than shown. Still, although shown as only including one of the adjustment mechanism 208, the valve 100 may include more than one of the adjustment mechanism 208, and in such instances, the adjustment mechanisms 208 may be collectively or individually actuatable.

The valve 100 includes biasing elements 214. In some instances, the biasing elements 214 are disposed between one or more ends of the bands 204, respectively. In some instances, the biasing elements 214 are a component of the base 202 and/or the adjustment mechanism 208. As shown, the valve 100 may include two of the biasing elements 214, such as a first biasing element 214(1) extending between the first band 204(1) and the third band 204(3), and a second biasing element 214(2) extending between the second band 204(2) and the third band 204(3). The biasing elements 214, as will be discussed herein, store mechanical energy and gradually release it over time to promote slow, continuous expansion of the valve 100, encouraging tissue remodeling at the implantation site. The leaflets 106 are designed with redundant coaptation to accommodate changes in the dimension of the valve 100.

The biasing elements 214 may include springs, elastic bands, etc. Although two of the biasing elements 214 are shown, the valve 100 may include more than or fewer than two of the biasing elements 214. Moreover, rather than including the biasing elements 214, the bands 204 may be manufactured from shape-memory materials or expandable polymers. Here, the biasing elements 214 may be omitted and the base 202 or the bands 204 may be designed to change geometry in response to specific stimuli such as temperature, pH, or light (e.g., UV activation). In some instances, the biasing element 214 may be omitted, and the size of the valve 100 may be adjusted via the adjustment mechanism 208. Alternatively, in some instances, the adjustment mechanism 208 may be omitted, and the size of the valve 100 may be adjusted via the biasing elements 214. Still, the valve 100 may incorporate both the adjustment mechanism 208 and the biasing elements 214.

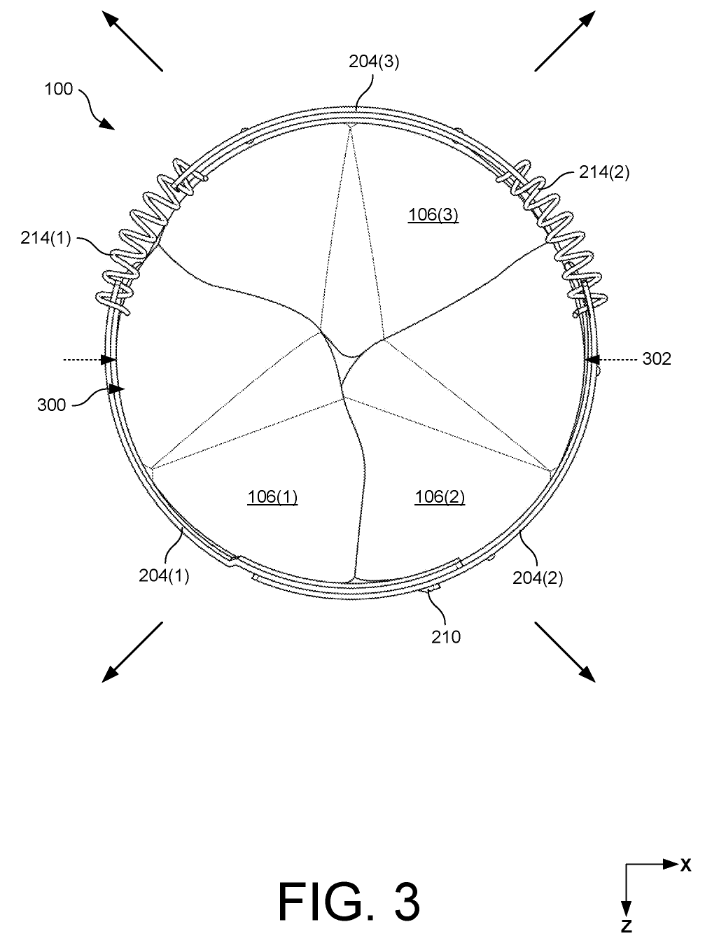

FIG. 3 illustrates an end view of valve 100, according to an example of the present disclosure. A first biasing element 214(1) is disposed between the first band 204(1) and the third band 204(3), while a second biasing element 214(2) is disposed between the second band 204(2) and the third band 204(3). During expansion of valve 100, such as by movement of the buckle 210 to a different one of the receptacles 212, by release of stored energy within the biasing elements 214, the base 202 undergoes radial outward expansion, as indicated by the arrows in FIG. 3. The frame 200, which is mechanically coupled to the base 202, expands accordingly. This radial expansion reduces the coaptation of the leaflets 106. Incorporating more than one of the biasing elements 214 around the circumference of the valve 100 may help promote uniform, symmetric expansion, improving deployment precision and minimizing stress concentrations on the frame 200 and the leaflets 106. This may also promote tissue growth within a native annulus of the patient.

The valve 100 includes an orifice 300 through which blood flows. The orifice 300 may be defined by the base 202, or more specifically, the bands 204. The orifice 300 has an internal diameter 302 that is adjusted (e.g., increased) during expansion of the valve 100. In some instances, as a size of the valve 100 is increased, the internal diameter 302 is also increased. In some instances, the internal diameter 302 is capable of being adjusted separately (e.g., through a separate adjustment mechanism).

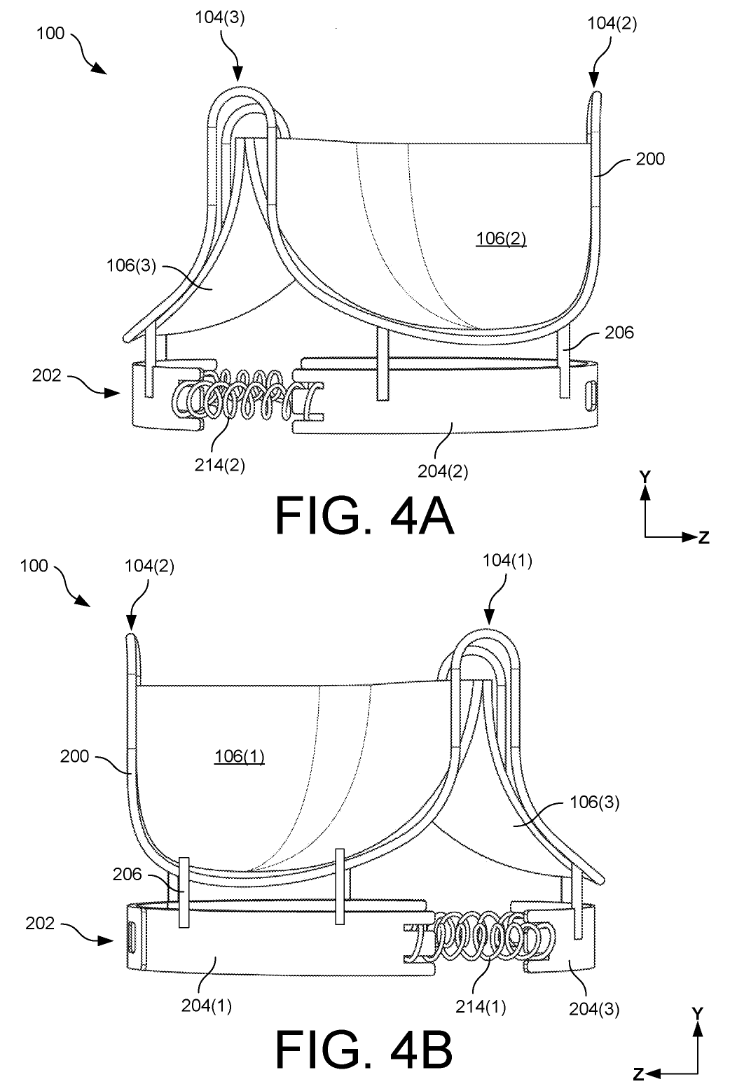

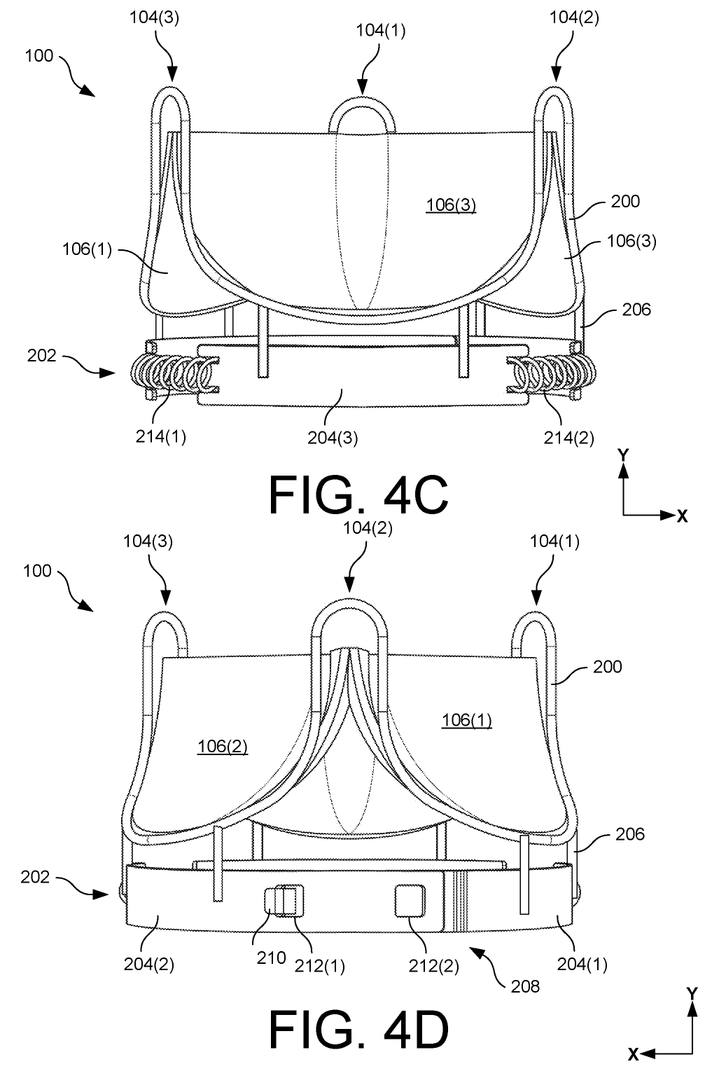

FIGS. 4A-4D illustrate various side views of the valve 100, showing the fabric 102 removed, according to an example of the present disclosure. The frame 200 secures to the bands 204, or more generally, the base 202, via the struts 206. In some instances, two of the struts 206 may extend between each of the bands 204 and the frame 200. The struts 206 may be oriented perpendicular to a lengthwise direction or the length of the bands 204. Alternatively, in some instances, the struts 206 may be oriented at other angles. Moreover, more than two of the struts 206 may be used to couple each of the bands 204 to the frame 200, and/or not all of the bands 204 may be coupled to the frame 200 via the struts 110. Although a specific shape or configuration of the bands 204 is shown, other variations are envisioned.

In some instances, the struts 206 are disposed between the frame 200 and the base 202 at a location interposed between adjacent posts 104. For example, a valley (e.g., a low point) may be disposed between adjacent peaks. Two of the struts 206 may be disposed between the first post 104(1) and the second post 104(2) to couple the frame 200 to the first band 204(1). Additionally, two of the struts 206 may be disposed between the second post 104(2) and the third post 104(3) to couple the frame 200 to the second band 204(2). Moreover, two of the struts 206 may be disposed between the first post 104(1) and the third post 104(3) to couple the frame 200 to the third band 204(3).

In some instances, the struts 206 may be rigid. Alternatively, the struts 206 may be flexible or semi-rigid, such as an elastic band, strap, etc. Regardless of the specific embodiment, the struts 206 may secure, mechanically couple, etc., the bands 204 to the frame 200 to transfer motion into the frame 200 during expansion of the adjustment mechanism 208. For example, the struts 206 may act on the frame 200 during adjustment of the adjustment mechanism 208 to urge the frame 200 outward and increase the size of the valve 100. In some instances, the struts 206 may be a component of the frame 200, the base 202, and/or the adjustment mechanism 208. The struts 206 may be coupled to the frame 200, the base 202, and/or the adjustment mechanism 208 via any suitable method, such as welding, adhesion, sewing, fastening, etc.

In some instances, although the frame 200 and the base 202 are illustrated as separate components, they may alternatively be manufactured as an integrated, unitary structure. In such instances, the frame 200 may be formed as a continuous extension of, or be directly affixed to, the bands 204 of the base 202, thereby reducing the number of discrete components. This direct connection may simplify manufacturing and assembly processes, enhance structural rigidity, and improve the consistency of force transmission throughout the valve system during actuation. Moreover, when the frame 200 and base 202 are integrated, the struts 206 may be eliminated. In some instances, integrating the frame 200 and base 202 may lead to better mechanical alignment and more predictable deformation patterns during expansion of the valve 100. However, such design choices may depend on application-specific factors, including material properties, loading conditions, and manufacturing capabilities.

The biasing elements 214 are disposed between one or more of the bands 204. The first biasing element 214(1) extends between the first band 204(1) and the third band 204(3). As will be explained herein, in some instances, the first band 204(1) and the third band 204(3) include slits, notches, grooves, etc., in which the first biasing element 214(1) is disposed to seat the first biasing element 214(1) on the first band 204(1) and the band 204(3), respectively. Similarly, the second biasing element 214(2) extends between the second band 204(2) and the third band 204(3) and may be disposed within slits, notches, grooves, etc., in the second band 204(2) and the third band 204(3) to seat the second biasing element 214(2) on the second band 204(2) and the third band 204(3), respectively.

During the expansion of the valve 100, the posts 104 may undergo a change in geometry, including a reduction in height (e.g., in the Y-direction). This height reduction occurs as the posts 104 are displaced radially outward, causing them to compress, deform, shorten, fold, etc., slightly along their vertical axis. A material of the frame 200 permits this vertical shortening. The decrease in the height of the posts 104 during the radial expansion of the valve 100 may also assist in maintaining engagement with the native annulus.

The base 202 includes the adjustment mechanism 208. Although described as a component of the base 202, in some instances, the adjustment mechanism 208 is separate from the base 202 and/or is a different component than the base 202. In some instances, the adjustment mechanism 208 includes the buckle 210 and the receptacles 212. As described above, the engagement between the buckle 210 and one of the receptacles 212 secures the first band 204(1) and the second band 204(2) together. As shown in FIG. 4D, for example, the buckle 210 may be disposed in the first receptacle 212(1). However, as will be explained herein, the buckle 210 may be disposed in the second receptacle 212(2) during an adjustment of the adjustment mechanism 208 to increase the size of the valve 100.

In some instances, the first biasing element 214(1) may be located vertically beneath, or substantially aligned with, the first post 104(1), thereby facilitating a direct and efficient transfer of expansion forces. Similarly, the second biasing element 214(2) may be positioned vertically beneath, or aligned with, the third post 104(3), creating a balanced configuration across the valve 100. The buckle 210 and the receptacles 212 may also be strategically aligned beneath the second post 104(2), positioning them at a central or intermediate location relative to the adjacent posts 104. The spatial arrangement of the biasing elements 214, buckle 210, and receptacles 212 ensures that the forces generated during expansion of the valve are evenly distributed across the frame 200, thereby minimizing asymmetrical loading and potential deformation.

For example, such alignment enables a more coordinated and simultaneous outward displacement of the posts 104, contributing to a uniform radial expansion of the valve 100. As the biasing elements 214 expand, they exert a force that is directly imparted into the posts 104, which in turn drives the controlled expansion of the valve 100. This optimized alignment not only enhances mechanical efficiency but also reduces the potential for localized stress concentrations that could compromise the structural integrity or sealing performance of the valve 100. It should be noted, however, that while this configuration offers improved performance, the biasing elements 214, buckle 210, and receptacles 212 are not limited to this specific arrangement and may be positioned differently based on design requirements, manufacturing constraints, or performance considerations.

In some instances, the leaflets 106 may be coupled along a portion of the frame 200, or the entirety of the frame 200. The leaflets 106 may be coupled to the frame 200 via adhesion, sewing, welding, etc. Moreover, as discussed above, the valve 100 may include the fabric 102 disposed at least partially around the frame 200 and the adjustment mechanism 208 to secure components together. In some instances, the fabric 102 may be sewn to the leaflets 106.

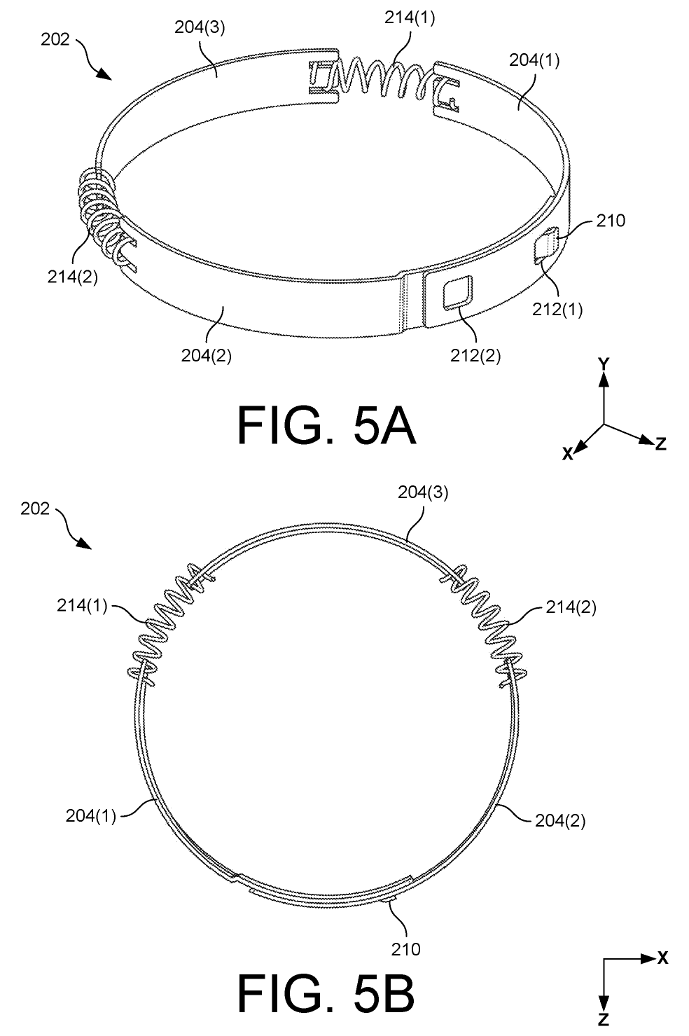

FIGS. 5A and 5B illustrate the base 202 and the adjustment mechanism 208, according to an example of the present disclosure. In some instances, the adjustment mechanism 208 may be structurally and/or functionally integrated with the base 202. For example, the adjustment mechanism 208 may be formed as a subcomponent or subassembly within the base 202, or the adjustment mechanism 208 may be co-manufactured as a monolithic unit. In such instances, the base 202 may serve not only as a structural foundation for supporting the frame 200 and the posts 104, but also as a mechanical interface through which actuation of the adjustment mechanism 208 is initiated and controlled.

In some instances, the base 202 may be defined as a broader assembly that includes the adjustment mechanism 208 as one of its functional elements. In such cases, the base 202 may encompass not only the bands 204 but also the components responsible for facilitating expansion of the valve 100, such as the buckle 210, the receptacles 212, etc. Similarly, the biasing elements 214 may be part of either the adjustment mechanism 208 or the base 202, depending on their integration. For instance, the biasing elements 214 may be embedded within the bands 204, secured along the inner circumference of the base 202, or operatively coupled to moving parts of the adjustment mechanism 208.

Alternatively, the adjustment mechanism 208 may be implemented as a distinct, modular component that is mechanically coupled to, but separate from, the base 202. In these instances, the adjustment mechanism 208 may be removably attached or permanently affixed to the base 202.

Introduced above, the base 202 includes the first band 204(1), the second band 204(2), and the third band 204(3). The first biasing element 214(1) is disposed between the first band 204(1) and the third band 204(3), and the second biasing element 214(2) is disposed between the second band 204(2) and the third band 204(3). The first band 204(1) and the second band 204(2) are secured together via the buckle 210 engaging with one of the receptacles 212. Moreover, the bands 204 are secured to one another (indirectly) via the struts 206 coupling to the frame 200.

In FIGS. 5A and 5B, the biasing elements 214 are shown in an expanded state. However, during actuation or repositioning of the buckle 210 to engage with a different one of the receptacles 212, the biasing elements 214 may be temporarily compressed. This compression stores potential energy within the biasing elements 214, which may be coil springs, elastic members, or shape-memory components, depending on the implementation. Once the buckle 210 is secured into the newly selected receptacle 212, the biasing elements 214 are allowed to gradually or immediately expand from their compressed state. As they expand, the biasing elements 214 impart a radial force outwardly through the connected posts 104 and bands 204, causing the overall structure of the valve 100 to enlarge.

This expansion results in an increase in the transverse dimensions of the valve 100, specifically an increase in the diameter of both the base 202 and the frame 200. The coordinated outward movement helps maintain geometric symmetry, which is important for proper sealing and anchoring within a surrounding annulus. The controlled nature of the expansion may be accomplished by different materials, spring constants, etc., to allow for fine-tuning of the deployed state of the valve 100 over time, which can be advantageous in dynamic anatomical environments where gradual adjustment is preferred.

Although described as including three of the bands 204, the base 202 may include more than or fewer than three of the bands 204. Additionally, or alternatively, although described as including two of the biasing elements 214, the base 202 may include more than or fewer than two of the biasing elements 214. The bands 204 themselves may include different or similar lengths, may be manufactured from different or similar materials, etc. The base 202 may also include structural members other than the bands 204, such as rings, struts, bars, etc.

Although the adjustment mechanism 208 is described herein as including the buckle 210 and a series of the receptacles 212 configured to incrementally modify the geometry of the valve 100, it should be understood that the adjustment mechanism 208 may alternatively or additionally include a wide variety of mechanical systems capable of effectively controlling expansion or contraction. For example, the adjustment mechanism 208 may incorporate ratcheting systems, gear-based actuators, screw/worm gear mechanism, threaded collars, or other unidirectional locking mechanisms that allow for stepwise or continuous modulation of the diameter of the valve 100. In some instances, the bands 204 may be configured with a series of axially spaced notches, teeth, or serrations that interface with a pawl, cam, or other locking fastener that may be selectively actuated. In one instance, the adjustment mechanism 208 may be similar to that of a hose clamp or cinch band, where rotation or linear translation of a fastener expands or contracts the base 202. For instance, a screw-driven band tightener or helical gear system may be used to apply circumferential force to the bands 204, increasing the diameter of the valve 100 in a precise and controlled manner. Alternatively, shape-memory components (e.g., Nitinol-based actuators) may be incorporated to produce temperature- or time-dependent expansion of the valve 100. Magnetically actuated or electromechanically driven components are also envisioned, allowing for remote or programmable size adjustment post-deployment. In all such cases, the adjustment mechanism 208 may be configured to enable reversible or irreversible changes to the geometry of the valve 100, depending on clinical requirements. These and other variations allow for a high degree of customization, enabling the valve 100 to adapt dynamically to patient-specific anatomy and to accommodate procedural adjustments before, during, or after implantation. Regardless of the specific embodiment, during actuation, whether manually via catheter-based delivery tools or automatically in response to physiological stimuli, the adjustment mechanism 208 is configured to incrementally expand the valve 100.

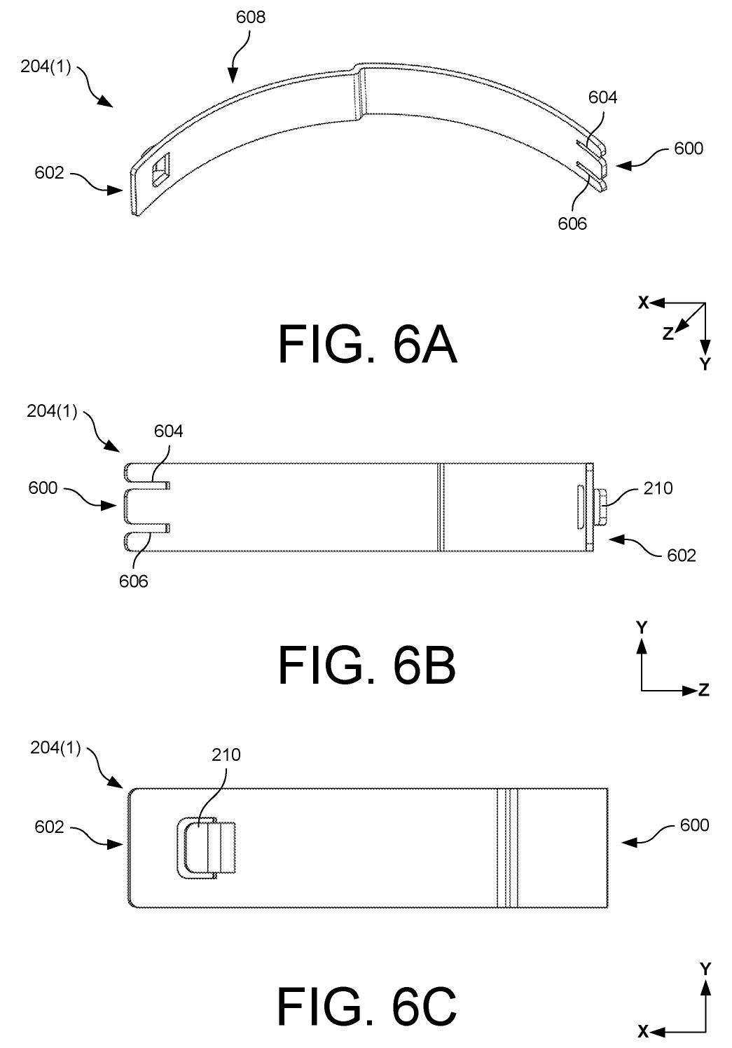

FIGS. 6A-6C illustrate various views of the first band 204(1), according to an embodiment of the present disclosure. The first band 204(1) may include a first end 600 and a second end 602 spaced apart from the first end 600. A length of the first band 204(1) extends between the first end 600 and the second end 602. As shown, the first band 204(1) may have a curved or arcuate shape, conforming to a portion of a circular geometry of the valve 100 or the base 202.

The first end 600 may define a first slot 604 and a second slot 606 for receiving a first end of the first biasing element 214(1). The first slot 604 and the second slot 606 may accommodate a diameter (e.g., wire diameter) of the first biasing element 214(1). Although a particular coupling or engagement between the first biasing element 214(1) is described, or the first band 204(1) is described as including certain features to receive the first biasing element 214(1), other variations are envisioned.

The buckle 210 may be disposed proximate to the second end 602. The buckle 210 may represent a portion of the first band 204(1) stamped, pumped, etc. For example, the buckle 1210 may present a flange, tab, rib, etc., extending from the first band 204(1). Although shown as including a certain configuration, the buckle 210 may be configured, shaped, sized, etc., differently. Moreover, although shown as including one buckle 210, the first band 204(1) may include more than one buckle 210. Still, the buckle 210 or alternative buckles may be located on alternate bands 204.

The first band 204(1) may include an indent 608 (e.g., pocket, groove, recess, channel, etc.) for accommodating the second band 204(2). For example, a portion of the first band 204(1) and the second band 204(2) may overlap, and the indent 608 may accommodate the overlap. The indent 608 may extend from the second end 602 in a direction towards the first end 600. The overlapping engagement may facilitate controlled adjustment of the valve 100 by allowing relative movement between the bands 204. The indent 608 may also serve to reduce the profile at the overlap region to minimizing bulk. The buckle 210 may be formed along a length of the first band 204(1) corresponding to the indent 608.

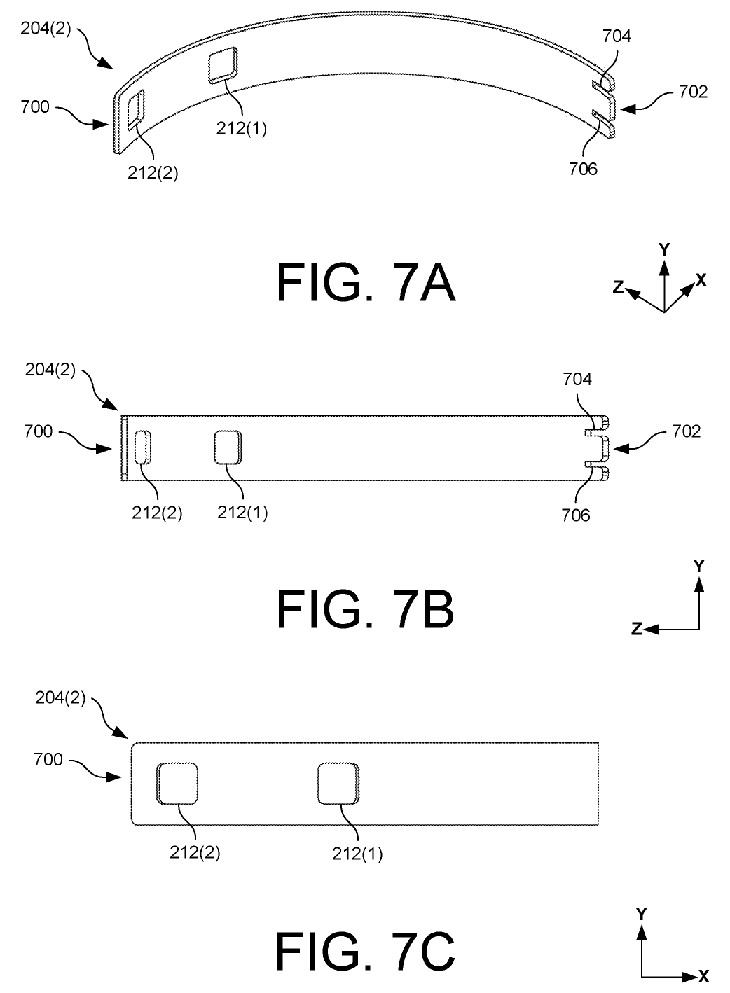

FIGS. 7A-7C illustrate various views of the second band 204(2), according to an embodiment of the present disclosure. The second band 204(2) may include a first end 700 and a second end 702 spaced apart from the first end 700. A length of the second band 204(2) extends between the first end 700 and the second end 702. As shown, the second band 204(2) may have a curved or arcuate shape, conforming to a portion of a circular geometry of the valve 100 or the base 202.

The second band 204(2) defines or includes the receptacles 212. In some instances, the receptacles 212 may be formed via stamping, punching, etc., portions of the second 204(2). As shown, the receptacles 212 are disposed proximate to the first end 700. In some instances, the receptacles 212 may be square-shaped. However, other shapes are envisioned. For example, a shape of the receptacles 212 may accommodate receipt of the buckle 210, as well as removal of the buckle 210, for example, during expansion of the valve 100. Regardless of the specific embodiment, the receptacles 212 may be shaped and configured to receive the buckle 210. Although shown and described as including two of the receptacles 212, the second band 204(2) may include more than or less than two of the receptacles 212, and the receptacles 212 may be located on the second band 204(2) differently than shown.

The second end 702 may define a first slot 704 and a second slot 706 for receiving a first end of the second biasing element 214(2). The first slot 704 and the second slot 706 may accommodate a diameter (e.g., wire diameter) of the second biasing element 214(2). Although a particular coupling or engagement between the second biasing element 214(2) is described, or the second band 204(2) is described as including certain features to receive the second biasing element 214(2), other variations are envisioned.

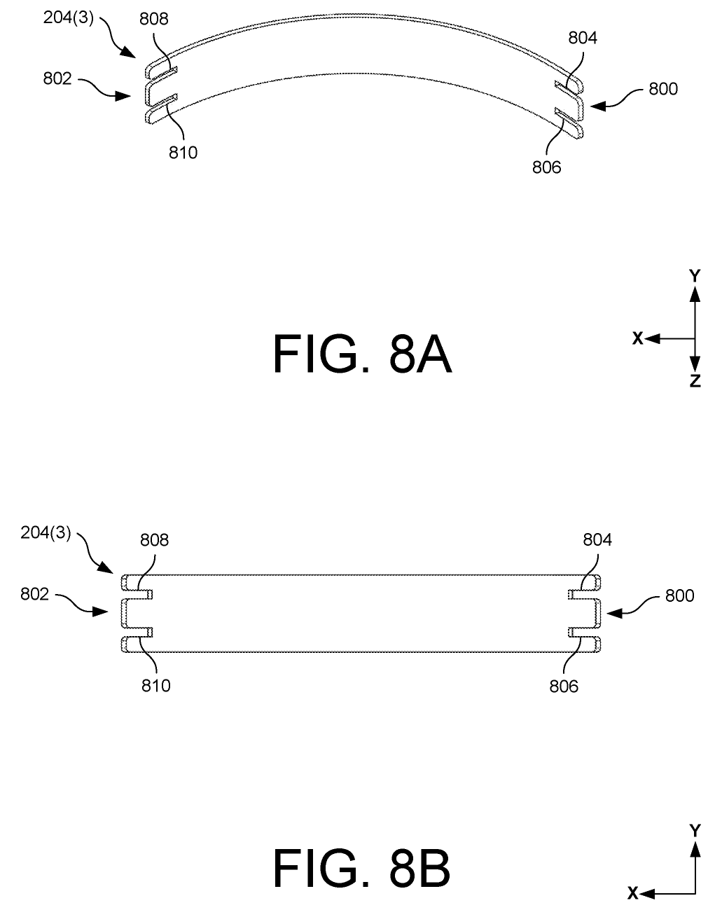

FIGS. 8A and 8B illustrate various views of the third band 204(3), according to an embodiment of the present disclosure. The third band 204(3) may include a first end 800 and a second end 802 spaced apart from the first end 800. A length of the third band 204(3) extends between the first end 800 and the second end 802. As shown, the third band 204(3) may have a curved or arcuate shape, conforming to a portion of a circular geometry of the valve 100.

The first end 800 may define a first slot 804 and a second slot 806 for receiving a second end of the first biasing element 214(1). The first slot 804 and the second slot 806 may accommodate a diameter (e.g., wire diameter) of the first biasing element 214(1). Likewise, the second end 802 may define a first slot 808 and a second slot 810 for receiving a second end of the second biasing element 214(2). The first slot 808 and the second slot 810 may accommodate a diameter (e.g., wire diameter) of the second biasing element 214(2).

Although a particular coupling or engagement between the first biasing element 214(1) is described, or the third band 204(3) is described as including certain features to receive the first biasing element 214(1), other variations are envisioned. Additionally, although a particular coupling or engagement between the second biasing element 214(2) is described, or the third band 204(3) is described as including certain features to receive the second biasing element 214(2), other variations are envisioned.

Although FIGS. 6A-6C, 7A-7C, and 8A and 8B illustrate certain details of the bands 204, respectively, other variations are envisioned. For example, more than one of the bands 204 may have a buckle (e.g., the buckle 210) that engages with one or more receptacles (e.g., the receptacles 212) disposed on another of the bands 204. In this sense, the valve 100 may have more than one of the attachment mechanisms 208, whereby multiple buckle-style attachment mechanisms may be used to expand and/or secure the size of the valve 100. In such instances, the bands 204 may be formed with respective buckles and receptacles, for example, in addition to or alternative from mechanisms for receiving the biasing elements 214.

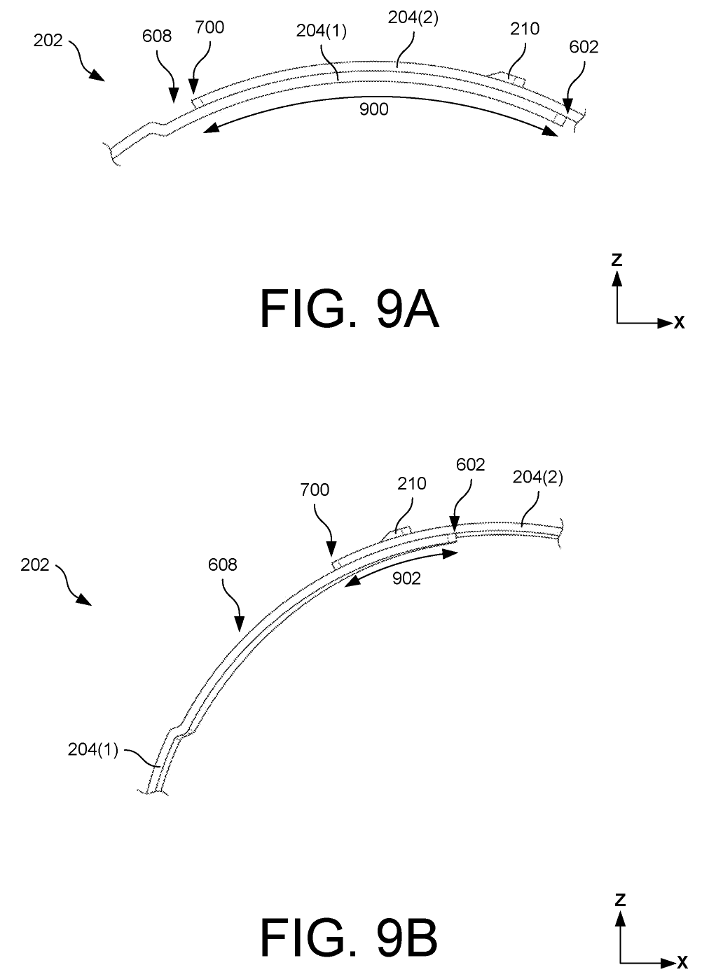

FIGS. 9A and 9B illustrate the overlapping configuration of the first band 204(1) and the second band 204(2), according to an example of the present disclosure. The overlapping interface enables incremental adjustment of the valve 100 by varying the relative positions of the bands 204 via engagement of the buckle 210 with different receptacles 212. In FIG. 9A, the buckle 210 is shown engaged with the first receptacle 212(1), representing an initial or less-expanded configuration of the valve 100. In this state, the first end 600 of the second band 204(2) is seated within the indent 608 formed in the outer or inner surface of the first band 204(1). This indent helps retain the overlapping portion of the second band 204(2) and maintains axial and radial alignment between the bands during movement. In this configuration, the first end 700 of the second band 204(2) is spaced apart from the second end 602 of the first band 204(1) by a first distance 900, representing a larger amount of overlap and a correspondingly smaller deployed diameter of the valve 100.

In contrast, FIG. 9B illustrates a subsequent configuration in which the buckle 210 is engaged with the second receptacle 212(2), corresponding to a further-expanded state of the valve. During this engagement, the first end 700 of the second band 204(2) remains disposed within the indent 608. However, the first end 700 is now spaced apart from the second end 602 of the first band 204(1) by a second distance 902, which is less than the first distance 900. This reduced overlap corresponds to a larger effective circumference and diameter of the base 202, thereby increasing the size of the valve 100.

This overlapping and sliding relationship between the bands 204 allows for controlled expansion of the valve 100 through discrete or continuous adjustment of the buckle 210 along a series of the receptacles 212.

During the expansion of the valve 100, the biasing elements 214 may undergo compression, such as when the buckle 210 moves from the first receptacle 212(1) to the second receptacle 212(2). This compression stores mechanical energy within the biasing elements 214, which may be in the form of coil springs, leaf springs, elastic components, or shape-memory alloys. Once the adjustment is secured, the biasing elements 214 (from a compressed state) begin to gradually release the stored energy, exerting a controlled, outward radial force on the surrounding structure, including the frame 200 and the base 202. This progressive release of energy facilitates a gradual and sustained expansion of the valve 100 over time. Moreover, this expansion may be particularly advantageous in reducing trauma to surrounding tissue, improving sealing performance, and enhancing long-term fit.

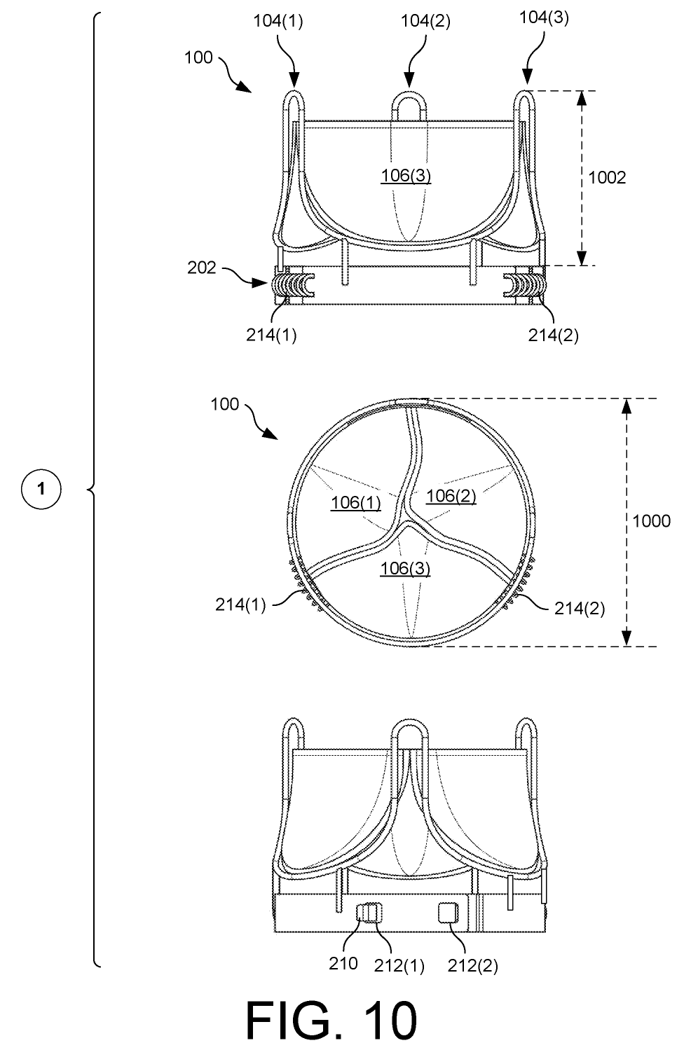

FIG. 10-13 illustrate an example progression to expand the valve 100, according to an example of the present disclosure. At “1” in FIG. 10, the valve 100, the base 202, or the adjustment mechanism 208 is shown having a first configuration (e.g., setting, size, etc.). In the first configuration, the valve 100 may have a first dimension 1000 (e.g., internal diameter, outer diameter, etc.). In the first configuration, the buckle 210 may be disposed in the first receptacle 212(1). In the first configuration, the biasing elements 214 may be in a compressed state. Moreover, in the first configuration, a peak (e.g., top) of the posts 104 may be disposed above the base 202 (e.g., a top of the bands 204) by a first distance 1002. In some instances, the first distance 1002 is associated with a height of the posts 104. In some instances, each of the peaks of the posts 104 may be disposed above the base 202 by the first distance 1002. As also shown, the leaflets 106 may have a redundant coaptation to accommodate future expansion of the valve 100.

In some instances, the valve 100 is initially restricted from expanding through a brace. For example, in the first configuration, a brace may be circumferentially disposed around the valve 100. The brace prevents expansion of the biasing elements 214, and correspondingly, the valve 100. However, upon or during installation into the native annulus, the brace may be removed. Therein, despite the buckle 210 not being moved from one of the receptacles 212, the valve 100 is permitted to expand, given that it’s no longer constricted by the brace. As such, the brace is used to hold the valve 100 in a compressed state, preventing any expansion. However, once installation is complete and the brace is removed, it no longer constrains the valve 100, allowing the biasing elements 214 to expand the valve 100.

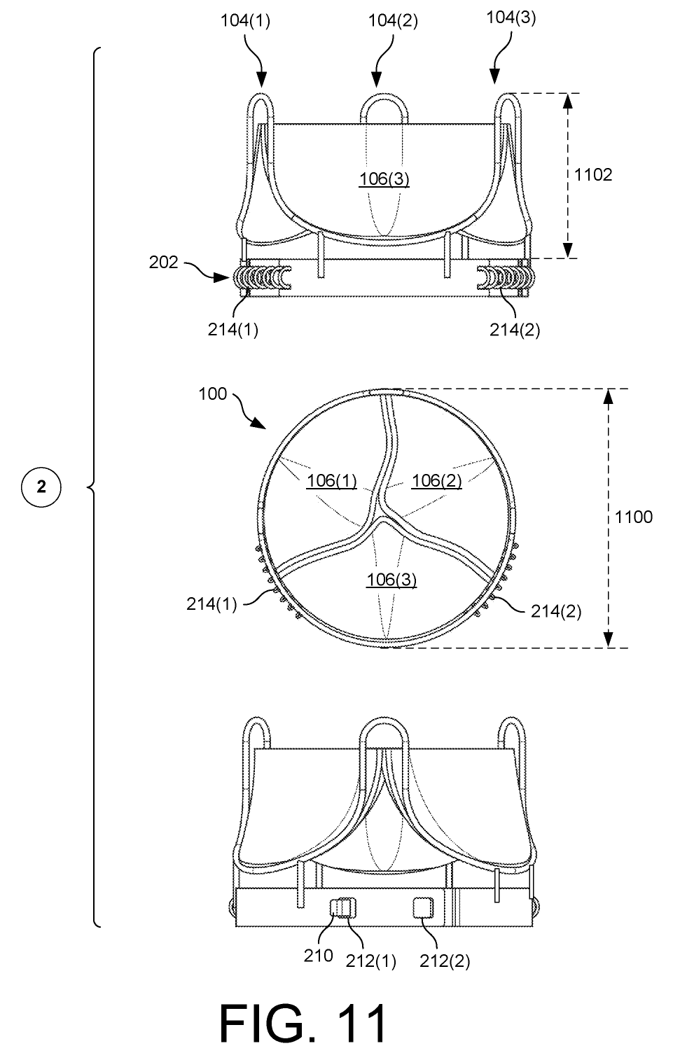

At “2” in FIG. 11, the valve 100 or the adjustment mechanism 208 is shown having a second configuration. In the second configuration, the valve 100 may have a second dimension 1100 (e.g., internal diameter, outer diameter, etc.) that is larger than the first dimension 1000. In the second configuration, the buckle 210 may be disposed in the first receptacle 212(1). In the second configuration, the biasing elements 214 may be in an expanded state. For example, although the buckle 210 still remains disposed within the receptacle 212(1), from “1” to “2”, the biasing elements 214 release energy to expand the valve 100. That is, the biasing elements 214 release energy into the base 202 and the frame 200, thereby causing the valve 100 to increase in size.

In the second configuration, the peak of the posts 104 may be disposed above the base 202 by a second distance 1102. The second distance 1102 may be less than the first distance 1002. As the valve 100 increases in size, and the frame 200 radially expands outwards, a height of the posts 104 is reduced. For example, as the frame 200 is urged outwards, the posts 104 are acted on and the posts 104 may flatten, so as to reduce in height. The material of the posts 104 permits such deformation. As also shown, the leaflets 106 may have a redundant coaptation to accommodate future expansion of the valve 100. The amount of redundant coaptation between “1” and “2” may be lessened given the expansion of the valve 100.

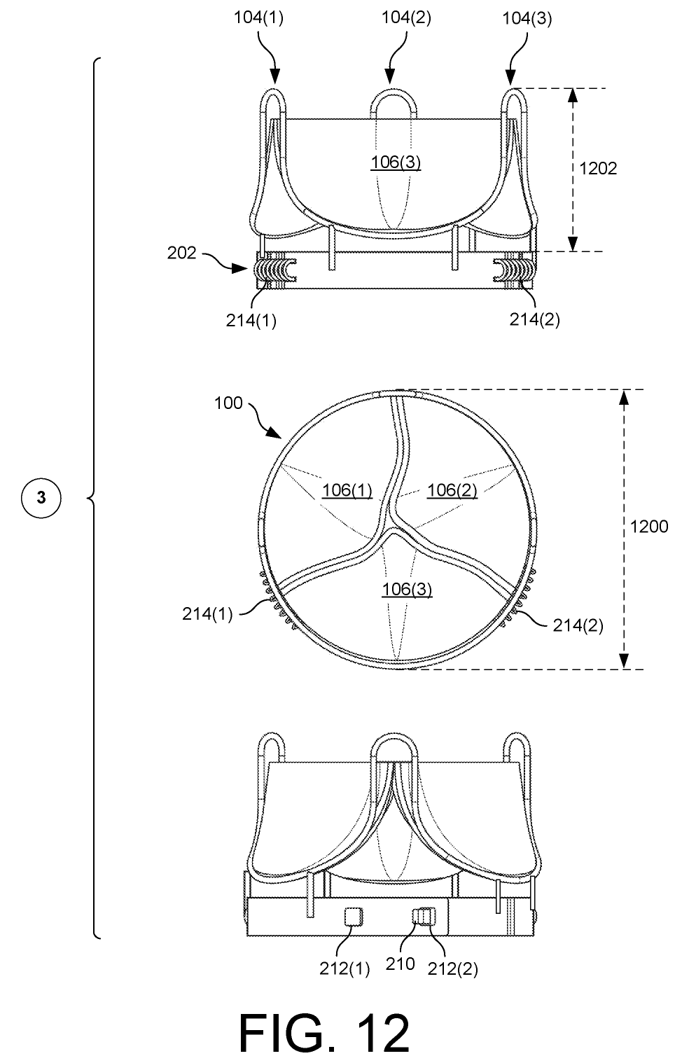

At “3” in FIG. 12, the valve 100 or the adjustment mechanism 208 is shown having a third configuration. In the third configuration, the valve 100 may have a third dimension 1200 (e.g., internal diameter, outer diameter, etc.) that is larger than the second dimension 1100. In the third configuration, the buckle 210 may be disposed in the second receptacle 212(2). For example, via a balloon or other mechanism (e.g., tool, instrument, magnets, etc.), the buckle 210 may be moved from the first receptacle 212(1) to the second receptacle 212(2). Movement of the buckle 210 to the second receptacle 212(2) causes an increase in the diameter of the valve 100.

Moreover, in the third configuration, the biasing elements 214 may be in a compressed state. That is, from “2” to “3”, the biasing elements 214 may be compressed for their expanded state. Because of the movement of the buckle 210 to the second receptacle 212(2), the biasing elements 214 may become compressed. Moreover, in the third configuration, the peak of the posts 104 may be disposed above the base 202 by a third distance 1202 that is less than the second distance 1102. For example, as the frame 200 is urged outwards and the posts 104 are acted on, the posts 104 may flatten, so as to reduce in height. The leaflets 106 at “3” may have a redundant coaptation to accommodate expansion of the valve 100. The amount of redundant coaptation between “2” and “3” may be lessened given the expansion of the valve 100.

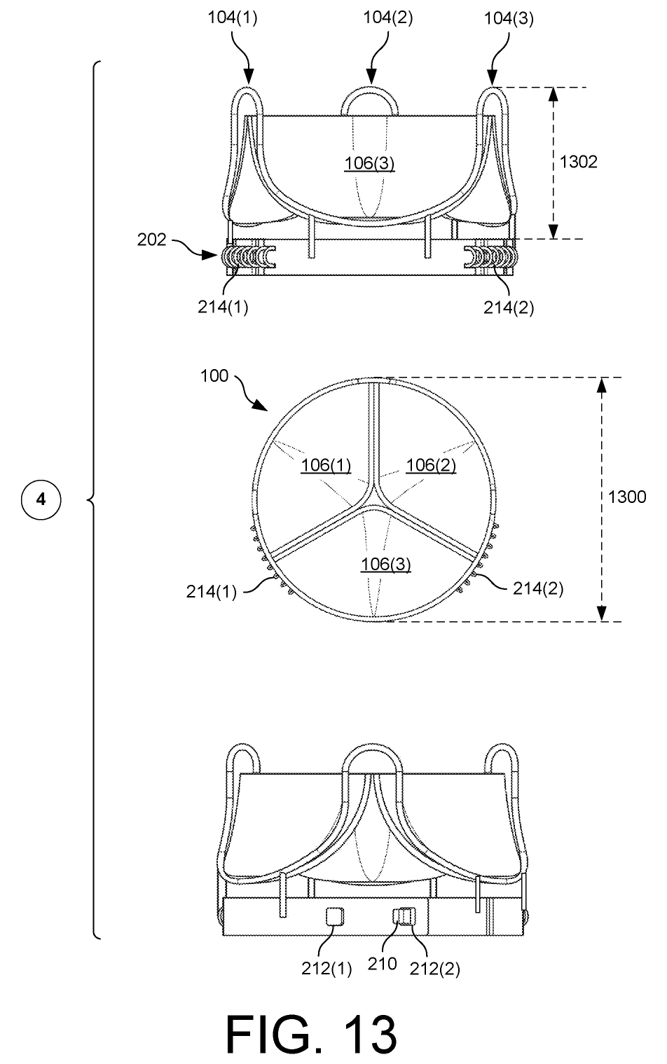

At “4” in FIG. 13, the valve 100 or the adjustment mechanism 208 is shown having a fourth configuration. In the fourth configuration, the valve 100 may have a fourth dimension 1300 (e.g., internal diameter, outer diameter, etc.) that is larger than the third dimension 1200. In the fourth configuration, the buckle 210 may be disposed in the second receptacle 212(2). In the fourth configuration, the biasing elements 214 may be in an expanded state. For example, although the buckle 210 remains disposed within the second receptacle 212(2), from “3” to “4”, the biasing elements 214 release energy to expand the valve 100. That is, the biasing elements 214 release energy into the frame 200, causing the valve 100 to expand.

Moreover, in the fourth configuration, the peak of the posts 104 may be disposed above the base 202 by a fourth distance 1302. The fourth distance 1302 may be less than the third distance 1202. As the valve 100 increases in size, and the frame 200 radially expands outwards, a height of the posts 104 is reduced. As also shown, the leaflets 106 may have an optimal coaptation at “4”. In some instances, a remaining amount of the redundant coaptation may be eliminated between “3” and “4”.

In some instances, characteristics of the valve 100 may be dependent upon the patient (e.g., native annulus), an initial size of the valve 100, and so forth. For example, the amount of redundant coaptation of the leaflets 106 at “1” in FIG. 10 may be based on the optimal coaptation, or the fourth dimension 1300 (i.e., as fully expanded). Moreover, a spring constant of the biasing elements 214, for example, a material of the biasing elements 214, a length of the biasing elements 214, a number of coils within the biasing elements 214, etc., may be adjusted to adjust the amount of radial force applied by the biasing elements 214, a size of the valve 100, and/or to accommodate the fourth dimension 1300.

Although FIGS. 10-13 illustrate the adjustment of the valve 100 from the first configuration to the fourth configuration, the valve 100 may have other configurations and/or the adjustment mechanism 208 may be adjusted to other configurations. For example, the adjustment mechanism 108 may be adjusted to an intermediate configuration between the first configuration and the second configuration. The intermediate configuration may assist in slowly expanding the size of the valve 100 to permit the tissue to accommodate increases in the size of the valve 100. As another example, the adjustment mechanism 208, or the second band 104(2), may have a third receptacle to which the buckle 210 is adjustable. Still, other types of adjustment mechanisms are envisioned. Regardless of the specific embodiment, through expansion of the valve 100, any number of steps or configurations may be used. In addition, the valve 100 may be increased either immediately after implantation or progressively over a period of time.

As another example, before the buckle 210 is disposed in the first receptacle 212(1) (e.g., at “1” in FIG. 10), the buckle 210 may be disposed external to the first receptacle 212(1). For example, at a time of implantation, or during delivery of the valve 100 to the annulus, the buckle 210 may be disposed external to the first receptacle 212(1). At this point in time, the buckle 210 may have its smallest size. During or after implantation, the valve 100 may be expanded by disposing the buckle 210 within the first receptacle 212(1). Here, the valve 100 may have a size larger than when implanted. This expansion, or moving the buckle 210 into engagement with the first receptacle 212(1), may be via the methods described above (e.g., balloon). From there, the valve 100 may be expanded at future instances in time, or may not be expanded at all depending upon the needs of the patient. As such, in some instances, the valve 100 may be expandable to two sizes, such as from first (initial) size to a second (later) size, or from more than two sizes.

Moreover, although discussed herein as increasing the size of the valve 100, in some instances, the size of the valve 100 may be decreased through operating the adjustment mechanism 108 in a reverse manner.

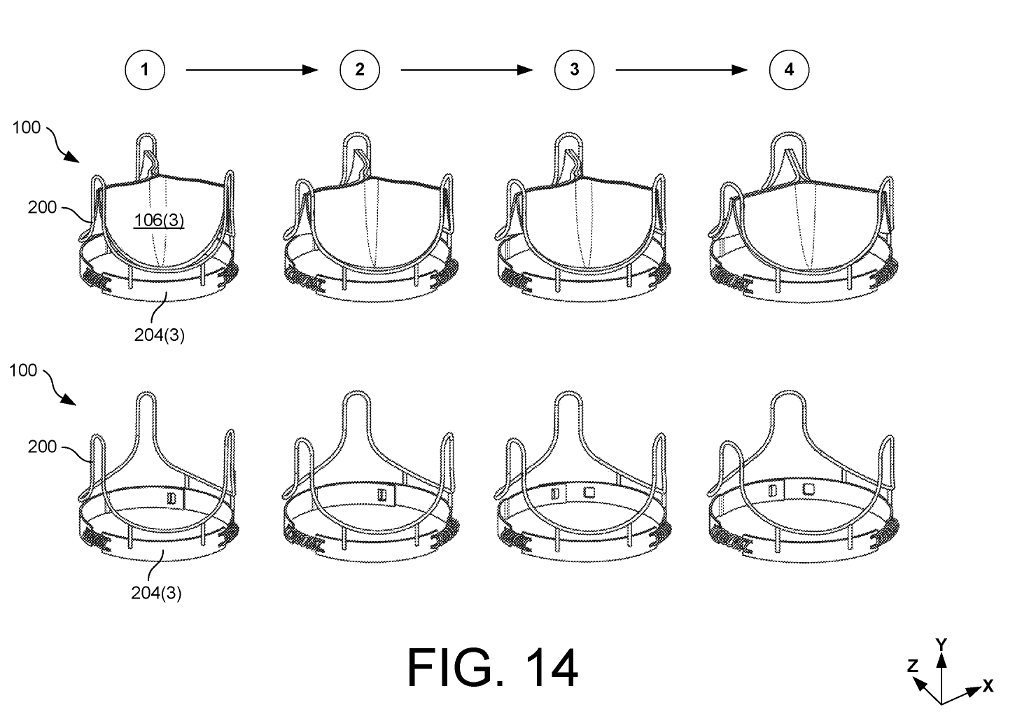

FIG. 14 illustrates an example sequence for expanding the valve 100, according to an example of the present disclosure. For example, “1” to “4” in FIG. 14 may correspond to “1” to “4” in FIGS. 10-13, respectively. During expansion of the valve 100, the frame 200 increases in dimension (e.g., diameter), simultaneously causing a shortening in the height of the posts 104 and/or a widening of the posts 104. Moreover, during the expansion of the valve 100, the redundant coaptation between the leaflets 106 is reduced.

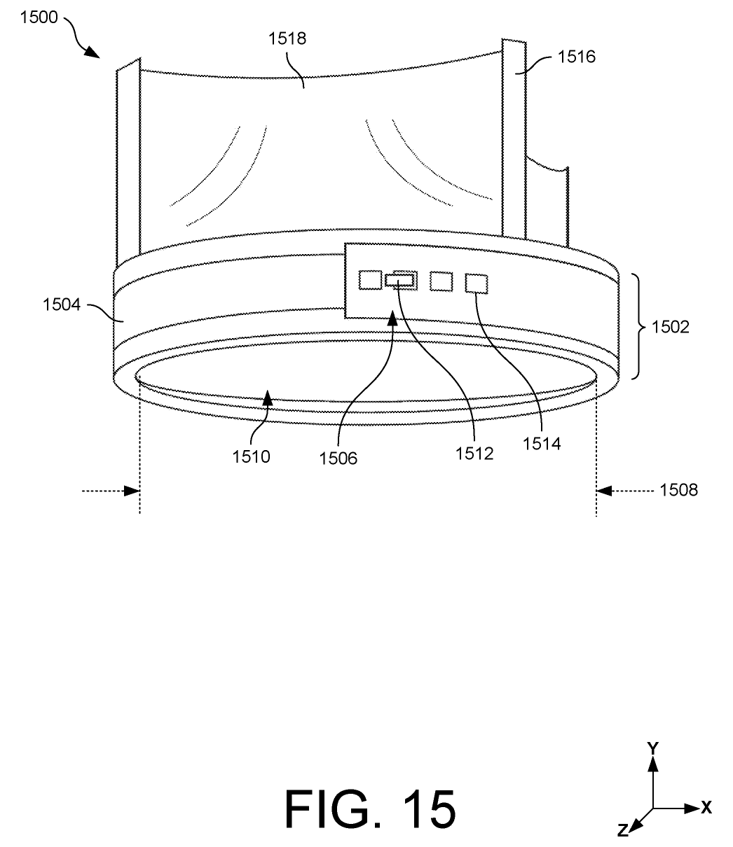

FIG. 15 illustrates an isometric view of an example valve 1500, according to examples of the present disclosure. In some instances, the valve 1500 may include a frame 1502 that is configured to radially expand outward after implantation into a patient or gradually over a period of time (e.g., along the X-Y plane). In some instances, the frame 1502 includes a band 1504 that is adjustable in size. An adjustment mechanism 1506 may secure the band 1504 together at different locations, depending upon the desired size of the valve 1500. Adjustment of the band 1504 may correspondingly adjust an internal diameter 1508 of an orifice 1510 defined by the frame 1502.

In some instances, the adjustment mechanism 1506 includes a clasp 1512 that engages with one or more receptacles 1514. The clasp 1512 and the receptacles 1514 may be formed in the band 1504. An engagement between the clasp 1512 and the receptacles 1514 may secure the band 1504 together, for example, to dictate a size of the valve 1500. The clasp 1512, however, is capable of being adjusted, so as to be disposed in different receptacles of the receptacles 1514, to adjust a size of the frame 1502 and correspondingly, the internal diameter 1508 of the orifice 1510. By way of example, the adjustment mechanism 1506 may be actuated (e.g., via tools, instruments, etc.) to move the clasp 1512 from a first of the receptacles 1514 to a second of the receptacles 1514. As the adjustment mechanism 1506 is adjusted the size of the frame 1502 correspondingly adjusts. In some instances, the frame 1502 is circular in shape. Although the valve 1500 is shown as including a particular adjustment mechanism 1506, other adjustment mechanisms, whether the same or different, may be used. For example, more than one adjustment mechanism may be used to adjust the size of the frame 1502 (or the orifice 1510). Additionally, although referred to herein as bands, the frame 1502 may include other struts, members, rings, etc., that operably engage with one another and that are adjustable. The frame 1502 may be made from a rigid, semi-rigid, or flexible material.

In some instances, the adjustment mechanism 1506 may be capable of expanding the internal diameter 1508 between 19 millimeters and 25 millimeters. However, other sizes are envisioned. Moreover, the internal diameter 1508 may be incrementally adjusted (e.g., in 2 mm increments), and in such instances, the band 1504 may include a predetermined number of the receptacles 1514 to accommodate the increased adjustments.

Although not shown, in some instances, the valve 1500 may include a biasing element (e.g., spring). In some instances, the biasing element may couple to the band 1504, or couple opposing ends of the band 1504 together. The biasing element may incrementally expand the frame 1502 by releasing energy. For example, the biasing element may represent a tension spring that is compressed during actuation of the adjustment mechanism 1506. As the adjustment mechanism 1506 expands the frame 1502, the biasing element becomes compressed. Over time, after adjustment of the adjustment mechanism 1506, the biasing element may release energy, thereby acting on the frame 1502, to cause the frame 1502 to expand further. This slow release of the frame 1502 may foster tissue growth within an annulus in which the valve 1500 is disposed.

The valve 1500 includes posts 1516 that extend from the frame 1502, such as the band 1504. During adjustment of the frame 1502, the posts 1516 may in turn be adjusted (e.g., expand radially outward). In some instances, the posts 1516 include three posts. Leaflets 1518 may extend between the posts 1516 and the frame 1502. In some instances, the valve 1500 includes three of the leaflets 1518. During the expansion of the frame 1502, given the coupling of the posts 1516 to the frame 1502, the leaflets 1518 may correspondingly be pulled and/or tightened. The leaflets 1518 may be designed with a redundant amount of coaptation to accommodate expansion of the frame 1502.

Although not shown, the band 1504, the adjustment mechanism 1506, the posts 1516, etc., may, in some instances, be at least partially encased, surrounded, etc., with fabric. Sutures may be disposed through the fabric and into the tissue of a patient to secure the valve 1500 within the patient.

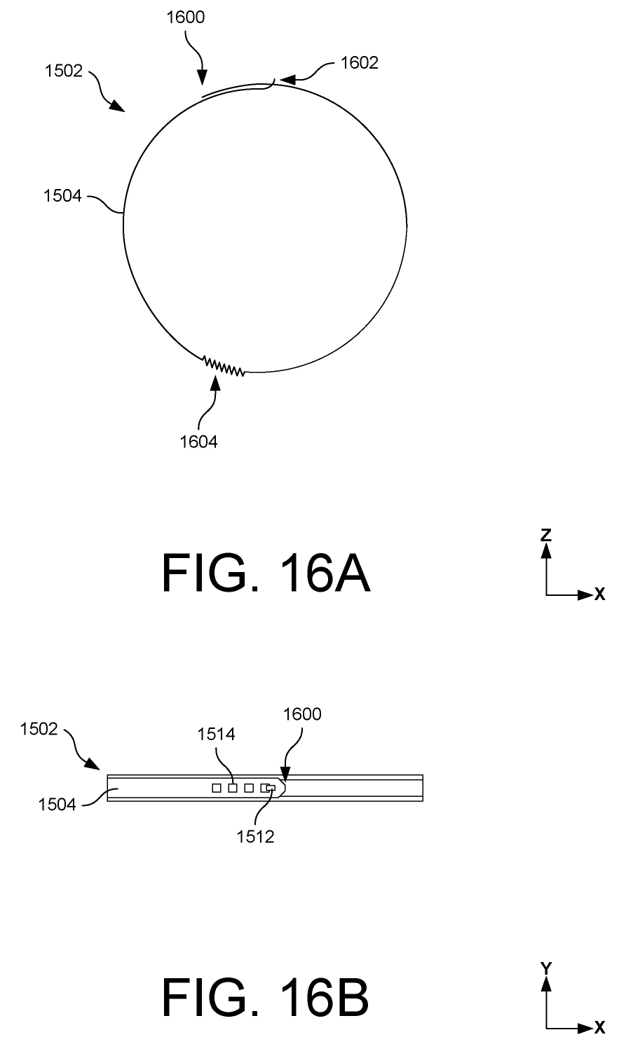

FIGS. 16A and 16B illustrate various views of the frame 1502, which includes the band 1504, according to an example of the present disclosure. In some instances, the band 1504 may be a continuous band or may be multiple bands coupled together. In some instances, the band 1504 includes a first end 1600 that is disposed through an opening of the band 1504, proximate to a second end 1602 of the band 1504. The band 1504 includes the receptacles 1514, and the second end 1602, which may include the clasp 1512, is configured to engage with one of the receptacles 1514. As shown, the first end 1600 may overlap with a portion of the band 1504. The overlap may accommodate adjustment of the band 1504 via the adjustment mechanism 1506. For example, when the band 1504 is expanded, the amount of overlap may be reduced.

The band 1504 may include a biasing element 1604, such as a spring (e.g., tension spring). The biasing element 1604 may be configured to expand the frame 1502. When the adjustment mechanism 1506 is adjusted to expand the band 1504, the biasing element 1604 may become compressed. The biasing element 1604 releases energy to incrementally expand the band 1504. Although the band 1504 is shown including a single biasing element, the band 1504 or the adjustment mechanism 1506 may include more than one biasing element. Characteristic(s) of the biasing element 1604, such as spring constant, material, number of coils, etc., may be adjusted to achieve the desired expansion of the frame 1502.

Although the adjustment mechanism 1506 is described as including the receptacles 1514 and the clasp 1512 that engages with the receptacles 1514, other variations of the adjustment mechanism 1506 are envisioned. For example, buckles, ratches, etc., may be used to adjust the frame 1502. In some instances, the band 1504 includes notches that engage with a fastener capable of being actuated. During actuation of the fastener, the band 1504 may be caused to expand (e.g., similar to a hose clamp, such as a screw/worm gear mechanism). This is just one example, and other variations of the adjustment mechanism 1506 are envisioned.

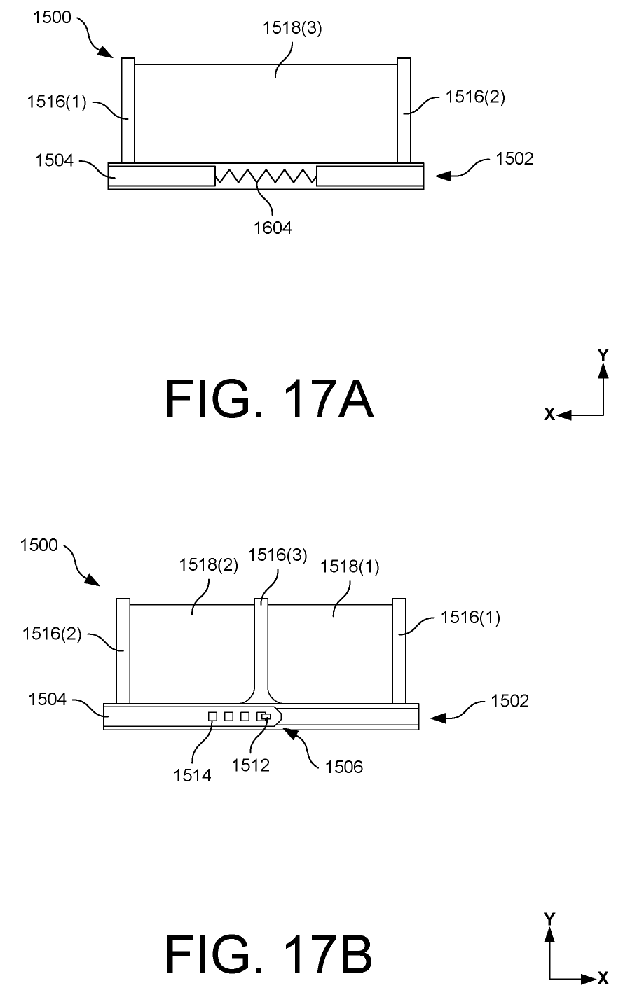

FIGS. 17A and 17B illustrate side views of the valve 1500, according to an example of the present disclosure. FIG. 17A illustrates a view of a first side of the valve 1500, and FIG. 17B illustrates a view of a second side of the valve 1500, opposite the first side. The valve 1500 includes the frame 1502, including the band 1504 and the posts 1516 that may be at least partially surrounded, wrapped, etc., with a fabric. In some instances, the adjustment mechanism 1506 may be integrally formed with the band 1504 or may be coupled to the band 1504.

The valve 1500 may include the first post 1516(1), the second post 1516(2), and the third post 1516(3). The first leaflet 1518(1) may extend between the first post 1516(1) and the second post 1516(2), the second leaflet 1518(2) may extend between the second post 1516(2) and the third post 1516(3), and the third leaflet 1518(3) may extend between the first post 1516(1) and the third post 1516(3). In addition, the leaflets 1518 may be coupled to the adjustment mechanism 1506 and/or the frame 1502 (e.g., the band 1504).

During adjustment of the adjustment mechanism 1506, for example, to expand the internal diameter 1508, the posts 1516 may be urged radially outward. During the radial expansion, the perimeter of the frame 1502 acts on the tissue of the patient (e.g., native annulus). As the band 1504 expands the posts 1516 are also urged outward. In addition, the leaflets 1518 are urged outward, thereby reducing the redundant coaptation during an increase in the internal diameter 1508. In instances in which the band 1504 includes multiple bands, the multiple bands may be coupled together via the biasing element 1604, or multiple biasing elements.

As shown in FIG. 17B, the adjustment mechanism 1506 may resemble a buckle. For example, the clasp 1512 (e.g., prong, hook, tab, etc.) may extend from the band 1504 and engage, or be disposed within, the receptacles 1514. When the adjustment mechanism 1506 is actuated, or otherwise adjusted, the clasp 1512 may be disposed in different receptacles 1514. An engagement between the clasp 1512 and one of the receptacles 1514 locks the band 1504 into place. However, upon application of a sufficient amount of force (e.g., via a balloon) or otherwise during actuation, the clasp 1512 may be moved to be disposed within another of the receptacles 1514, thereby increasing the internal diameter 1508.

In some instances, the transcatheter balloon may act on the frame 1502 during inflation. As the transcatheter balloon is pressed, or otherwise urged, against the frame 1502, the adjustment mechanism 1506 permits the frame 1502 to expand. For example, the clasp 1512 may release from a first receptacle and engage with a second receptacle, whereby movement of the clasp 1512 from the first receptacle to the second receptacle causes the frame 1502 to expand. In some instances, a predetermined amount of force may be required to release the clasp 1512 from the first receptacle and engage with the second receptacle, and/or other receptacles during expansion. The transcatheter balloon may be deflated and removed when the clasp 1512 is engaged with the second receptacle. However, the engagement between the clasp 1512 and the second receptacle retains the frame 1502 in position to resist contraction.

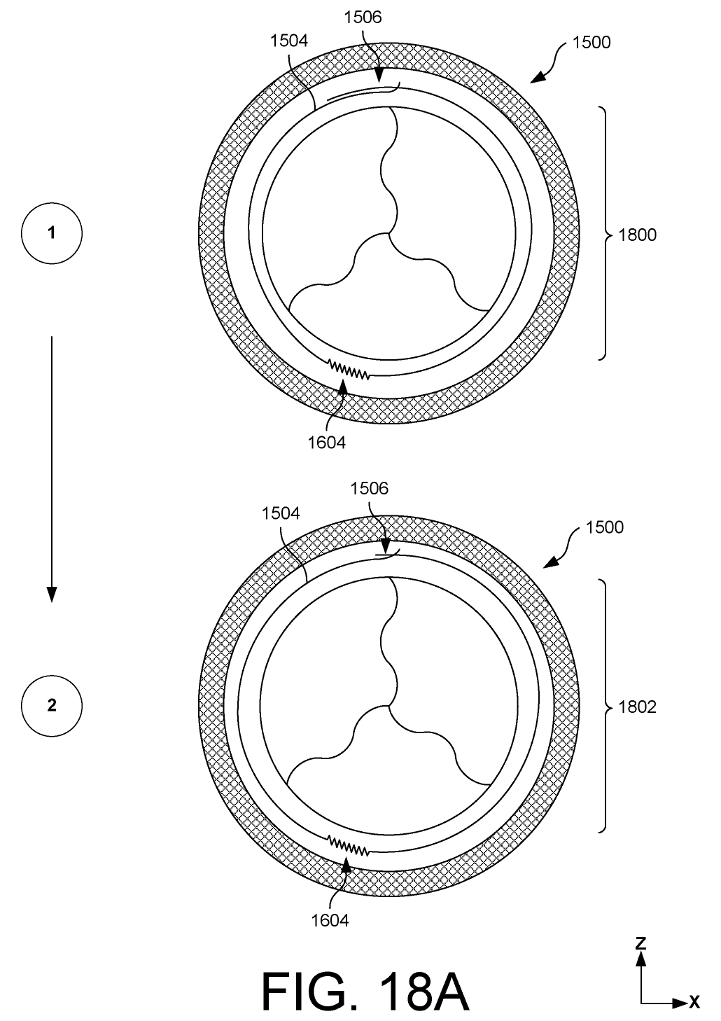

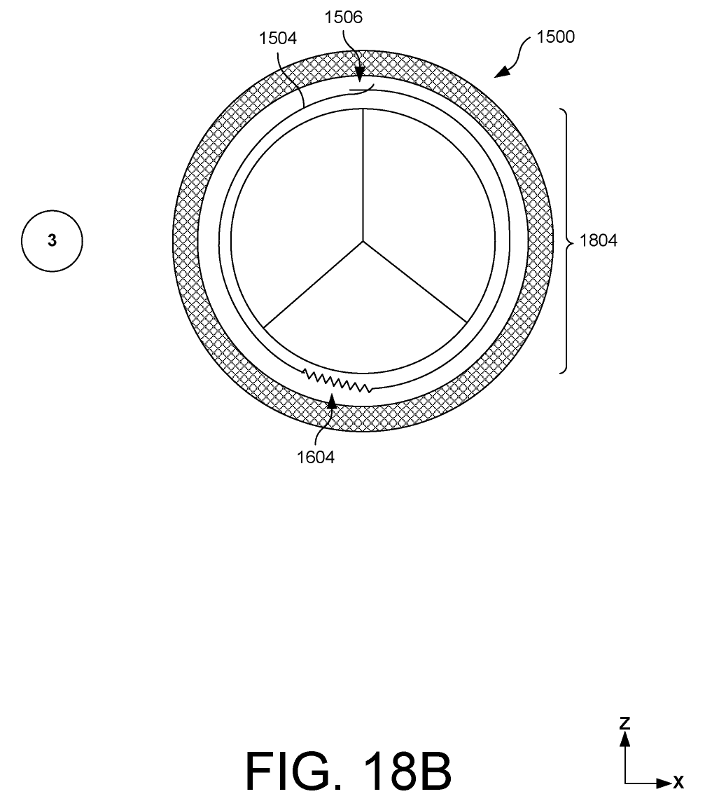

FIGS. 18A and 18B illustrate an example sequence associated with adjusting the valve 1500, according to examples of the present disclosure. Initially, at “1” in FIG. 18A, the frame 1502 may be in a compressed configuration. For example, the adjustment mechanism 1506 may be at a first setting, position, configuration, etc. At the first setting, the leaflets 1518 are shown having a redundant coaptation to permit expansion during adjustment of the valve 1500. In some instances, the depiction of the valve 1500 at “1” in FIG. 18A may be at the implantation of the valve 1500. At “1” in FIG. 18A, the valve 1500 may have a first internal diameter 1800. The first internal diameter 1800 may be the smallest diameter of the valve 1500 at the time of implantation. In some instances, at “1” in FIG. 18A, the biasing element 1604 may be in a non-compressed state or a compressed state. For example, the biasing element 1604 may be in a compressed state via being restricted from a brace during implantation. Regardless of the specific scenario, the valve 1500 may be expanded via the adjustment mechanism 1506 or through the biasing element 1604 releasing energy.

At “2” in FIG. 18A, the valve 1500 may be in a compressed configuration via the adjustment mechanism 1506 moving from the first setting to a second setting, position, configuration, etc. At the second setting, the band 1504 acts on the biasing element 1604 to compress the biasing element 1604. In other words, as the band 1504 expands, the band 1504 compresses the biasing element 1604. As shown, at the second setting, an amount of overlap of the band 1504 is lessened. The adjustment mechanism 1506 may be actuated to increase the frame 1502 after the valve 1500 is secured within the annulus.

The adjustment mechanism 1506 increases the internal diameter 1508 of the valve 1500, for example, to a second internal diameter 1802 that is greater than the first internal diameter 400. Moreover, compressing of the biasing element 1604 causes the biasing element 1604 to slowly expand over a period of time. For example, slowly, the biasing element 1604 may expand to exert a force on the band 1504. Stated alternatively, the frame 1502 may be loaded with tension, via the biasing element 1604, that causes the frame 1502 to slowly expand over time. As the force is exerted on the band 1504, the internal diameter 1508 undergoes radial expansion. The slow exertion of the force by the biasing element 1604 allows the tissue to adapt to increases in the internal diameter 1508 of the valve 1500. That is, as the internal diameter 1508 expands, the tissue slowly accommodates an increase to the internal diameter 1508. In this sense, the internal diameter 1508 may be expanded at a first instance in time upon adjustment of the adjustment mechanism 1506. Over a second period of time, however, the biasing element 1604 causes the internal diameter 1508 to slowly expand.

Adjustment of the adjustment mechanism 1506, from the first setting to the second setting, may come by way of tools, instruments, magnets, electronics, or other widgets/gadgets. As an example, a tool may be used to move the clasp 1512 from a first of the receptacles 1514 to a second of the receptacles 1514. The adjustment mechanism 1506 may be adjusted intravascular or extravascular.

At “3” in FIG. 18B, the valve 1500 is shown being fully expanded. The adjustment mechanism 1506 may not be adjusted between “2” in FIG. 18A and “3” in FIG. 18B, but the biasing element 1604 is shown being expanded (e.g., in length). As the biasing element 1604 expands, introduced above, the biasing element 1604 exerts a force that causes the internal diameter 1508 to increase. For example, at “3” in FIG. 18B, the valve 1500 may have a third internal diameter 1804 that is greater than the second internal diameter 1802. As also shown at “3” in FIG. 18B, the leaflets 1518 have achieved optimal coaptation. As the internal diameter 1508 expands, the leaflets 1518 are correspondingly expanded to optimal coaptation. Moreover, the biasing element 1604, in the expanded state, may no longer apply a radial force to the valve 1500 that causes the valve 1500 to expand. However, at future instances, the adjustment mechanism 1506 may be further adjusted. When fully expanded, the internal diameter 1508 effectuates improved hemodynamics and transcatheter valve-in-valve procedures.

In some instances, characteristics of the valve 1500 may be dependent upon the patient, an initial size of the internal diameter 1508 of the valve 1500, and so forth. For example, the amount of redundant coaptation of the leaflets 1518 at “1” in FIG. 18A may be based on the optimal coaptation, or the third internal diameter 1804 (i.e., as fully expanded). Moreover, when the biasing element 1604 is a spring, a spring constant of the spring, a material of the spring, a length of the spring, a number of coils within the spring, etc., may be adjusted to adjust the amount of radial force to accommodate the third internal diameter 1804.

Although FIGS. 18A and 18B illustrate an adjustment of the adjustment mechanism 1506 from the first setting to the second setting, the adjustment mechanism 1506 may be adjusted more than once. For example, the adjustment mechanism 1506 may be adjusted to an intermediate setting between the first setting and the second setting. The intermediate setting may assist in slowly expanding the internal diameter 1508 to permit the tissue to accommodate increases to the internal diameter 1508. Through expansion of the internal diameter 1508, any number of steps or settings may be used. In addition, the internal diameter 1508 may be increased either immediately after implantation or progressively over a period of time. Immediate expansion may maximize the diameter of the annulus, while a longer period of progressive expansion may allow the tissue to slowly expand and remodel over time.

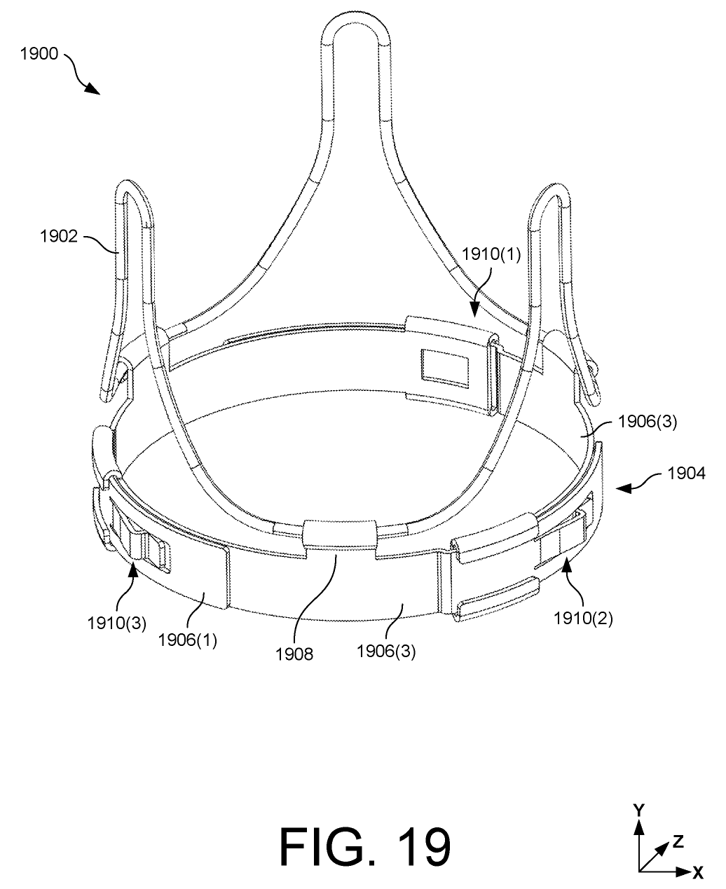

FIG. 19 illustrates an example valve 1900, according to an example of the present disclosure. In some instances, the valve 1900 may be similar to the valve 100 as discussed hereinabove. For example, the valve 1900 may include a frame 1902 having one or more posts (e.g., similar to the posts 104) that secure leaflets. Similar to the frame 200, the frame 1902 is configured to expand during adjustment of the valve 1900. The frame 1902 is secured to a base 1904, or bands 1906 that form the base 1904. As shown, the base 1904 may include three of the bands 1906, such as a first band 1906(1), a second band 1906(2), and a third band 1906(3). The base 1904, or the bands 1906, may be secured to the frame 1902 via struts 1908. In some instances, the struts 1908 are flanges, protrusions, lips, etc., that extend from the bands 1906, or which are integral to the bands 1906. Moreover, the struts 1908 may form receptacles, channels, etc., in which a portion of the frame 1902 is received. Regardless of the specific embodiment, the struts 1908 serve to couple the base 1904 to the frame 1902.

In some instances, the valve 1900 includes more than one adjustment mechanism, or the adjustment mechanism may include more than one interlocking points, points of connection, etc. For example, the valve 1900 may include adjustment mechanisms 1910, such as a first adjustment mechanism 1910(1), a second adjustment mechanism 1910(2), and/or a third adjustment mechanism 1910(3). The first adjustment mechanism 1910(1) may be formed at, or by, an engagement between the first band 1906(1) and the second band 1906(2), the second adjustment mechanism 1910(2) may be formed at, or by, an engagement between the second band 1906(2) and the third band 1906(3), and the third adjustment mechanism 1910(3) may be formed at, or by, an engagement between the first band 1906(1) and the third band 1906(3). Similar to the discussion above, and as will be explained herein, the adjustment mechanisms 1910 may be adjusted via various methods (e.g., a balloon). During the adjustment, each of the adjustment mechanisms 1910 may be actuated or adjusted synchronously or asynchronously to alter the size of the valve 1900. In some instances, each of the adjustment mechanisms 1910 include a buckle that engages with one or more receptacles.

In FIG. 19, the valve 1900 is shown having a first size. In some instances, the first size of the valve 1900 may be associated with a smallest size of the valve 1900. In some instances, at the first size, the adjustment mechanisms 1910 may not be engaged, or stated alternatively, the buckles of the adjustment mechanisms 1910 may not be disposed within the receptacles. However, after or during a time of implantation, the valve 1900 may be expanded such that the buckles of the adjustment mechanisms 1910 are disposed within the receptacles, respectively. Post implantation, at a later ViV procedures, the valve 1900 may be expandable to additional sizes (e.g., a third size, fourth size, etc.,) depending upon the number of receptacles, for example. In some instances, the initial size of the valve 1900 is 21mm.

Although not shown, the valve 1900 may include a fabric disposed over the frame 1902, the base 1904, etc. Moreover, the valve 1900 may include leaflets having redundant coaptation before implantation, and optimal coaptation after implantation. Although not shown, the valve 1900 may additionally or alternatively include biasing elements.

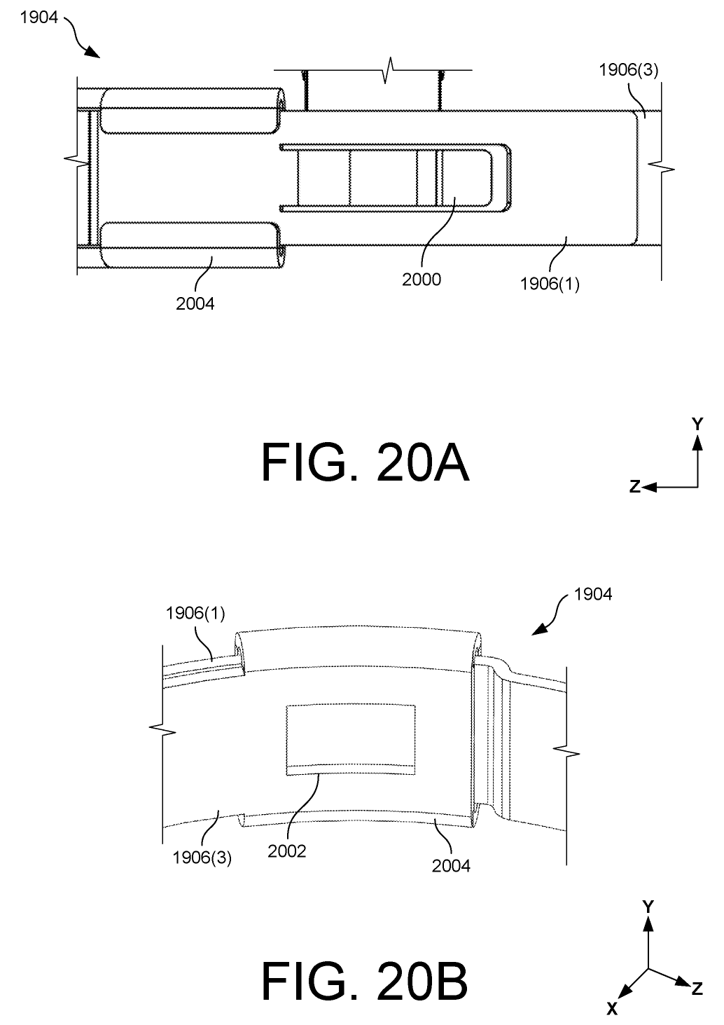

FIGS. 20A and 20B illustrate details of the third adjustment mechanism 1910(3), according to an example of the present disclosure. The third adjustment mechanism 1910(3) may be formed from, or by, the first band 1906(1) and the third band 1906(3). The first band 1906(1) may include, or form, a buckle 2000. The buckle 2000 is configured to be disposed within a receptacle 2002 formed by the third band 1906(3). As shown in FIGS. 20A and 20B, the buckle 2000 is shown not disposed within, or engaged with, the receptacle 2002. However, during expansion of the valve 1900, for example, via a transcatheter balloon, the buckle 2000 may move into the receptacle 2002. When disposed within the receptacle 2002, the valve 1900 may have an increased size.

In some instances, the buckle 2000 may be disposed external to the receptacle 2002 prior to, or during, implantation. In this instance, the valve 1900 may have a first size (e.g., 21mm). In some instances, once disposed within the native annulus, the buckle 2000 may be moved to the receptacle 2002 during an expansion of the valve 1900. In some instances, the valve 1900 may be expanded to 25mm. Although shown as only including one of the receptacles 2002, the third band 1906(3), or more generally, the bands 1906, may include additional receptacles to accommodate future expansion of the valve 1900. The valve 1900 may also include biasing elements in addition or, or in the alternative from, the adjustment mechanisms 1910.

The first band 1906(1) and the third band 1906(3) are configured to overlap in nature. In some instances, the third band 1906(3) may include flanges 2004 that engage with an exterior surface (or side) of the first band 1906(1). This may maintain an engagement between the first band 1906(1) and the third band 1906(3) as the base 1904 is expanded, and/or to accommodate the radial expansion of the base 1904.

Although the discussion herein is with regard to the third adjustment mechanism 1910(3), the first adjustment mechanism 1910(1) and the second adjustment mechanism 1910(2) may be similar, function similarly, etc. For example, the first band 1906(1) may include a receptacle for receiving a buckle of the second band 1906(2), and the second band 11906(2) may include a receptacle for receiving a buckle of the third band 1906(3). In some instances, the adjustment mechanisms 1910 may be actuated synchronously or asynchronously. For example, the adjustment mechanisms 1910 may be actuated to expand the valve 1900 from 21mm to 25mm.

Although the valve 1900 is described as including three of the adjustment mechanisms 1910, in some instances, the buckles and receptacles disposed across the bands 1906 may be referred to as single adjustment mechanism, where the single adjustment mechanism may include multiple interlocking or engagement points disposed across the bands 1906. Other variations of the adjustment mechanisms are envisioned. Moreover, although a buckle-style adjustment mechanism is described herein, it should be understood that a wide variety of alternative mechanisms are envisioned within the scope of this disclosure. The term “buckle” is used for convenience and may encompass other forms such as tabs, flanges, clasps, hooks, or similar engagement features capable of securing and adjusting the valve’s size. These mechanisms may include, but are not limited to, ratcheting systems, locking clasps, expanding latches, gear-based actuators, or other mechanical interlocks. The adjustment mechanism 1910 may be implemented in any configuration that provides secure, reliable, and controllable expansion or contraction of the valve, whether actuated manually, mechanically, or by other means.

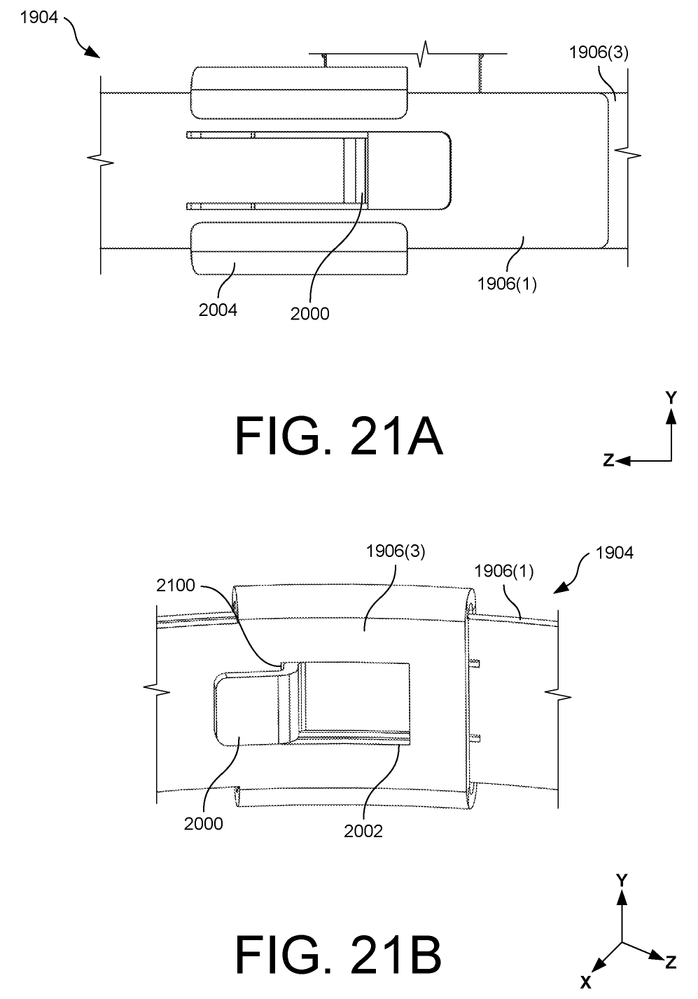

FIGS. 21A and 21B illustrate the buckle 2000 disposed within the receptacle 2002, according to an example of the present disclosure. For example, from the size of the valve 1900 as shown in FIGS. 20A and 20B, the valve 1900 may be expanded via adjustment mechanisms 1910. For example, as shown in FIGS. 21A and 21B, the buckle 2000 may be disposed within the receptacle 2002. In some instances, the buckle 2000 may engage within a sidewall 2100 of the receptacle 2002. This engagement may ensure that the valve 1900 is expanded to its proper size. Adjustment of the valve 1900 to the expanded size may come by way of a transcatheter balloon, during which the transcatheter balloon urges against the bands 1906 and the bands 1906 move in sliding relationship to one another. During movement of the bands 1906, the buckle 2000 becomes disposed within the receptacle 2002 and engages with the sidewall 2100. A disposition of the first band 1906(1) within the flanges 2004 maintains the engagement.

Although shown as only including one receptacle, the bands 1906 may include additional receptacles to accommodate future expansion of the valve 1900 after implantation. Moreover, although the discussion herein is with regard to the third adjustment mechanism 1910(3), the first adjustment mechanism 1910(1) and the second adjustment mechanism 1910(2) may be similar, function similarly, etc.

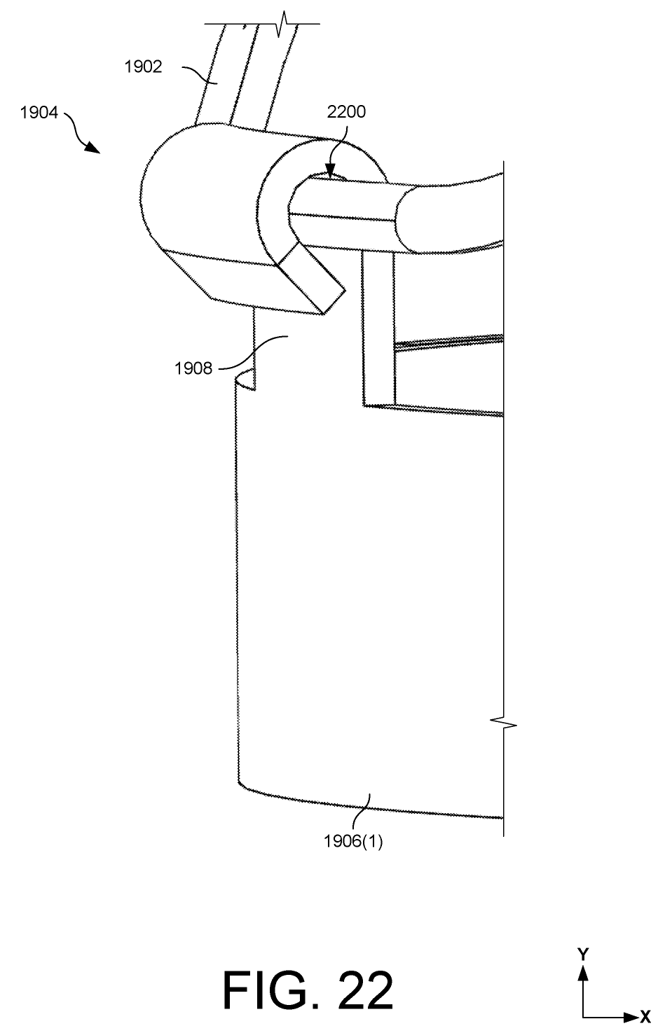

FIG. 22 illustrates an example coupling between the base 1904 and the frame 1902, according to an example of the present disclosure. The struts 1908 may couple the base 1904 to the frame 1902. In some instances, each of the bands 1906 that form the base 1904 may couple to the frame 1902 via more than one of the bands 1906. The struts 1908 may be formed as a flange, lip, projection, etc., that extends from the bands 1906, respectively. In some instances, the strut 1908 defines a receptacle 2200 in which the frame 1902 is disposed. The frame 1902 may be configured to slide within the receptacle 2200 during expansion of the valve 1900. However, as the base 1904 is expanded, the frame 1902 may be correspondingly by expanded via a coupling to the base 1904.

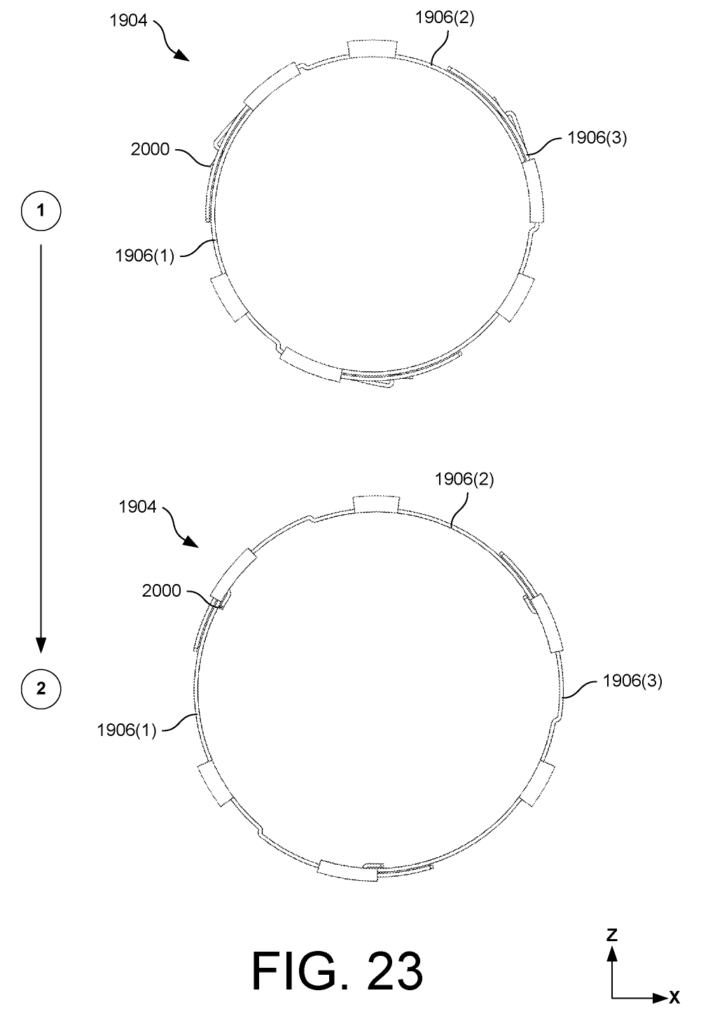

FIG. 23 illustrates the base 1904, according to an example of the present disclosure. The base 1904 includes the first band 1906(1), the second band 1906(2), and the third band 1906(3). The base 1904, or more generally, the valve 1900 may include the first adjustment mechanism 1910(1), the second adjustment mechanism 1910(2), and the third adjustment mechanism 1910(3). As shown at “1” in FIG. 23, the base 1904 may have a first size, whereby the buckle 2000 of the adjustment mechanisms 1910 are disposed external to the receptacle 2002. At “2” in FIG. 23, the base 1904 may have a second size, whereby the buckle 2000 is disposed internal, or within, the receptacle 2002.

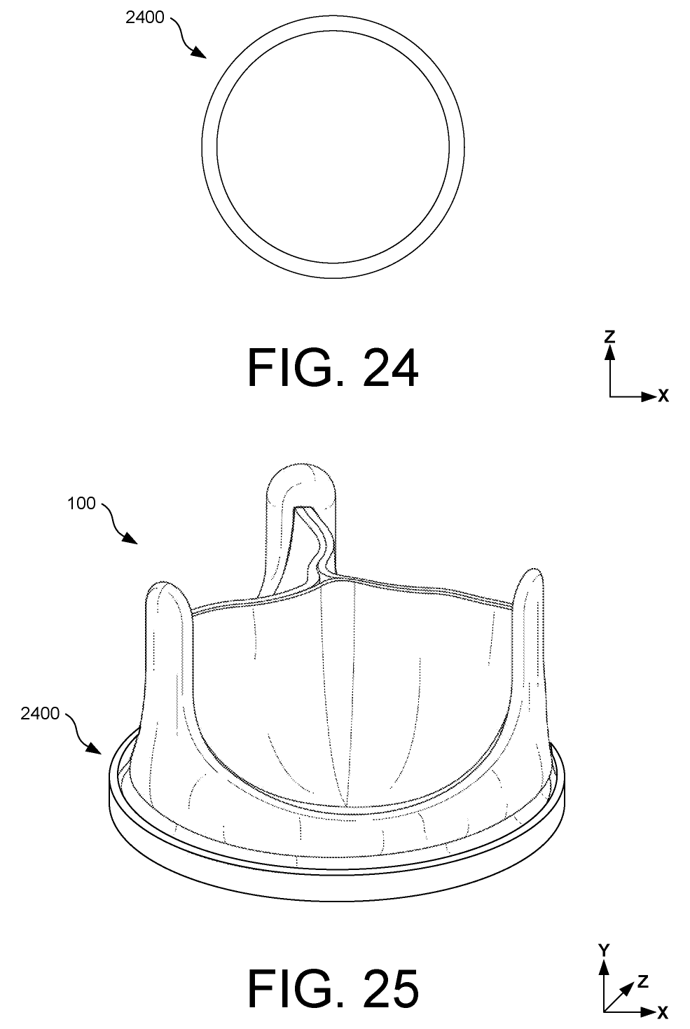

FIG. 24 illustrates an example brace 2400 that may be disposable around the valve 100, the valve 1500, and/or the valve 1900, according to an example of the present disclosure. The brace 2400, which may be alternatively referred to or represent a retainer, apparatus, etc., may be positioned circumferentially around the valve 100, the valve 1500, and/or the valve 1900 to temporarily restrict or prevent expansion, particularly during delivery, positioning, or implantation of the valve 100, the valve 1500, and/or the valve 1900 within a patient. The brace 2400 may be disposed around a structural portion of the valve 100, the valve 1500, and/or the valve 19000 such as a base, frame, or bands, and serves to counteract the force exerted by the biasing elements. As discussed above, these biasing elements are configured to drive the valve 100, the valve 1500, and/or the valve 1900 from a collapsed to an expanded configuration. By constraining these biasing elements, the brace 2400 maintains the valve 100, the valve 1500, and/or the valve 1900 in a reduced-profile, unexpanded state, which facilitates easier delivery into the patient.

In some instances, the brace 2400 may be constructed from a variety of biocompatible materials, including metals, polymers, or composites, and may be rigid, semi-rigid, or flexible, depending on the desired application. In some instances, the brace 2400 may take the form of a clamp, sleeve, ring, or cage, and may be secured using mechanical fasteners, friction fit, thermal bonding, or adhesive. The brace 2400 may be disposable or reusable and may be designed to be removed post-implantation via mechanical actuation, dissolution, or cutting, such as using a catheter-delivered cutting tool or surgical instrument. Once removed, the brace 2400 ceases to constrain the valve 100, the valve 1500, and/or the valve 1900, allowing the biasing elements to drive expansion and enable the valve 100, the valve 1500, and/or the valve 1900 to deploy into its fully functional, expanded configuration.

FIG. 25 illustrates the brace 2400 being disposed around the valve 100, according to an example of the present disclosure. Although described in conjunction with the valve 100, the brace 2400 may be usable with the valve 1500 and/or the valve 1900. As shown, the brace 2400 may be disposed around the base 202 of the valve 100 to constrict, or prevent, expansion of the biasing elements 214. Once implanted, or prior to implantation, the brace 2400 may be removed (e.g., cut) to allow the valve 100 to be sutured to the patient and to allow the biasing elements 214 to expand. In some instances, the brace 2400 may be used to reduce a profile of the valve 100 during implantation and/or to prevent premature expansion of the biasing elements 214.

While various examples and embodiments are described individually herein, the examples and embodiments may be combined, rearranged, and modified to arrive at other variations within the scope of this disclosure.

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims.

What is claimed is:

1. A valve comprising:
a base configured to expand from a first state to a second state, the base including a first band having a buckle, a second band having a receptacle, wherein in the first state, the buckle is disposed external to the receptacle, and in the second state, the buckle is disposed internal to the receptacle;
a frame coupled to the base; and
leaflets coupled to the frame.

2. The valve of claim 1, wherein the frame includes one or more posts coupled to the leaflets.

3. The valve of claim 1, further comprising a fabric disposed at least partially around the frame, the base, and the leaflets.

4. The valve of claim 1, further comprising one or more biasing elements.

5. The valve of claim 1, wherein:
the first band includes a second receptacle;
the second band includes a second buckle;
in the first state, the second buckle is disposed external to the second receptacle; and
in the second state, the second buckle is disposed internal to the second receptacle.

6. The valve of claim 1, further comprising one or more struts that couple the base to the frame, respectively.

7. A valve comprising:
a base;
a frame including:
a first post,
a second post, and
a third post;
leaflets including:
a first leaflet engaged with the first post and the second post,
a second leaflet engaged with the first post and the third post, and
a third leaflet engaged with the second post and the third post; and
an adjustment mechanism that adjusts a size of the valve from a first cross-sectional size to a second cross-sectional size, the second cross-sectional size being greater than the first cross-sectional size.

8. The valve of claim 7, wherein:
at the first cross-sectional size, the first post, the second post, and the third post have a first height disposed above the base; and
at the second cross-sectional size, the first post, the second post, and the third post have a second height disposed above the base, the second height being less than the first height.

9. The valve of claim 7, wherein;
at the first cross-sectional size, the leaflets have a first redundant coaptation; and
at the second cross-sectional size, the leaflets have a second redundant coaptation, the second redundant coaptation being less than the first redundant coaptation.

10. The valve of claim 7, wherein the frame couples to the base via one or more struts, bands, or linkages.

11. The valve of claim 7, wherein:
the base includes at least a first band and a second band; and
a biasing element is engaged with the first band and the second band.

12. The valve of claim 7, wherein the adjustment mechanism is at least one of disposed on, coupled to, or integrated with the base.

13. The valve of claim 7, wherein the adjustment mechanism is at least one of a buckle-style adjustment mechanism, a ratchet-style adjustment mechanism, or a worm screw gear mechanism.

14. The valve of claim 7, further comprising a second adjustment mechanism that adjusts the size of the valve from the first cross-sectional size to the second cross-sectional size.

15. A valve comprising:
a base adjustable from a first cross-sectional size to a second cross-sectional size, the base including a first band having a first buckle and a first receptacle, and a second band having a second buckle and a second receptacle disposed, wherein:
in the first cross-sectional size, the first buckle is disposed external to the second receptacle and the second buckle is disposed external to the first receptacle, and
in the second cross-sectional size, the first buckle is disposed internal to the second receptacle and the second buckle is disposed internal to the first receptacle;
a frame coupled to the base including:
a first post,
a second post, and
a third post; and
leaflets including:
a first leaflet engaged with the first post and the second post,
a second leaflet engaged with the first post and the third post, and
a third leaflet engaged with the second post and the third post.

16. The valve of claim 15, further comprising one or more biasing elements.

17. The valve of claim 16, wherein the one or more biasing elements adjust the valve to a third cross-sectional size.

18. The valve of claim 15, wherein the first buckle, the first receptacle, the second buckle, and the second receptacle are components of one or more adjustment mechanisms of the valve.

19. The valve of claim 15, wherein the base includes a third band.

20. The valve of claim 19, wherein:
in the first cross-sectional size, the first band and the second band have a first amount of overlap; and
in the second cross-sectional size, the first band and the second band have a second amount of overlap that is smaller than the first amount of overlap.
